(19) United States
(12) Patent Application Publication
(10) Pub. No.: US 2006/0052627 A1
(43) Pub. Date: Mar. 9, 2006

(54) Novel modulators of the PPAR-type receptors and cosmetic/pharmaceutical compositions comprised thereof

(75) Inventors: Philippe Diaz, Nice (FR); Etienne Thoreau, Saint Vallier De Thiey (FR); Isabelle Carlavan, Grasse (FR); Pascale Mauvais, Antibes (FR); Johannes Voegel, Chateauneuf/Grasse (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA RESEARCH & DEVELOPMENT, S.N.C., VALBONNE SOPHIA ANTIPOLIS (FR)

(21) Appl. No.: 11/202,019

(22) Filed: Aug. 12, 2005

(30) Foreign Application Priority Data
Feb. 10, 2004 (WO) PCT/EP04/02198
Feb. 12, 2003 (FR) FR 03/50025

(51) Int. Cl. C07D 241/02; C07D 239/02; C07D 237/02; C07C 229/40

(52) U.S. Cl. 560/129; 544/224; 544/406; 544/335; 546/335

(57) ABSTRACT

Novel compounds which modulate PPAR-type receptors have the following general formula (I): 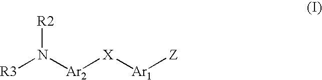 and are formulated into pharmaceutical compositions suited for administration in human or veterinary medicine (in dermatology and in the fields of cardiovascular diseases, immune diseases and/or diseases linked to lipid metabolism), or are formulated into cosmetic compositions.

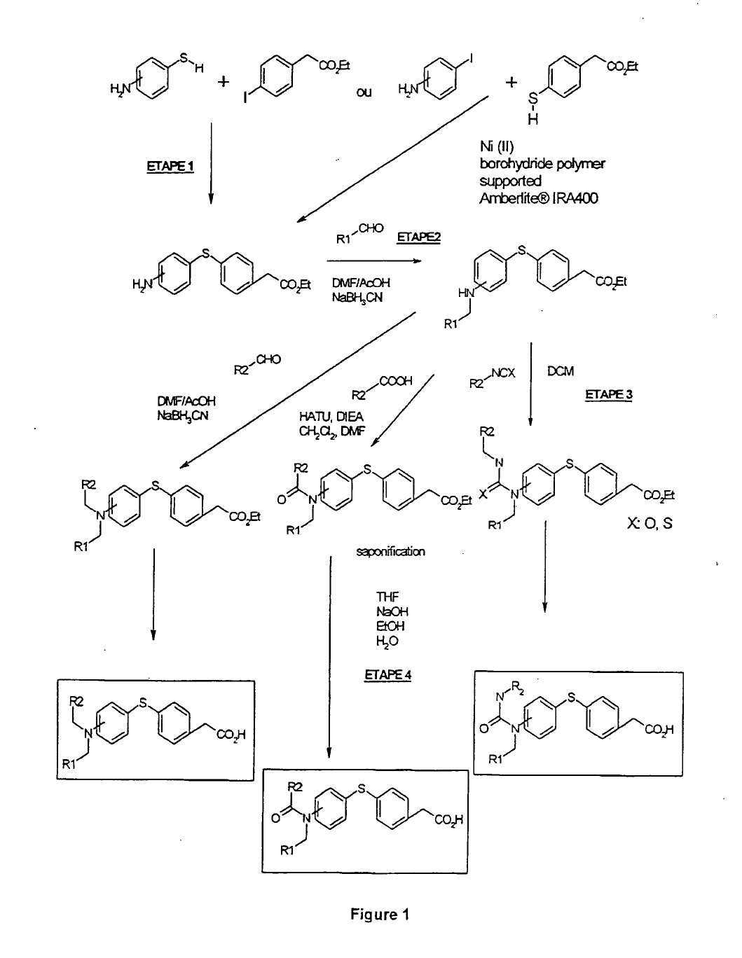

### CROSS-REFERENCE TO PRIORITY/PCT/PROVISIONAL APPLICATIONS

This application claims priority under 35 U.S.C. § 119 of FR 03/50025, filed Feb. 12, 2003, and of provisional application Ser. No. 60/453,835, filed Mar. 12, 2003, and is a continuation of PCT/EP 2004/002198, filed Feb. 10, 2004 and designating the United States (published in the English language on Aug. 26, 2004 as WO 2004/071504 A1), each hereby expressly incorporated by reference and each assigned to the assignee hereof.

### CROSS-REFERENCE TO COMPANION APPLICATION

Copending application Ser. No. ______ [Attorney Docket No. 034227-593], filed concurrently herewith and also assigned to the assignee hereof.

### BACKGROUND OF THE INVENTION

1. Technical Field of the Invention

The present invention relates, as novel and useful industrial products, to a novel class of compounds which are modulators of the Peroxisome Proliferator-Activated Receptor (PPAR) type receptors. It also relates to their method of preparation and to their formulation into pharmaceutical compositions for administration in human or veterinary medicine, or alternatively in cosmetic compositions.

2. Description of Background and/or Related and/or Prior Art

The activity of the PPAR-type receptors has been the subject of numerous studies. There may be mentioned, as a guide, the publication entitled “Differential Expression of Peroxisome Proliferator-Activated Receptor Subtypes During the Differentiation of Human Keratinocytes”, Michel Rivier et al., J. Invest. Dermatol., 111, 1998, p. 1116-1121, in which a large number of bibliographic references relating to PPAR-type receptors is listed. There may also be mentioned, as a guide, the dossier entitled “The PPARs: From orphan receptors to Drug Discovery”, Timothy M. Willson, Peter J. Brown, Daniel D. Sternbach, and Brad R. Henke, J. Med. Chem., 2000, Vol. 43, p. 527-550.

The PPAR receptors activate transcription by binding to elements of DNA sequences, called peroxisome proliferator response elements (PPRE), in the form of a heterodimer with the retinoid X receptors (known as RXRs).

Three human PPAR subtypes have been identified and described: PPARα, PPARγ and PPARδ (or NUC1).

PPARα is mainly expressed in the liver while PPARδ is ubiquitous. It is described in WO 98/32444 that PPARα selective compounds play a role in the barrier function and the differentiation of the stratum corneum.

PPARγ is the most widely studied of the three subtypes. All the references suggest a critical role of the PPARγ receptors in the regulation of differentiation of adipocytes, where it is highly expressed. It also plays a key role in systemic lipid homeostasis.

It has in particular been described in WO 96/33724 that PPARγ-selective compounds, such as prostaglandin-J2 or -D2, are potential active agents for treating obesity and diabetes.

### SUMMARY OF THE INVENTION

A novel class of PPAR-modulating compounds has now been developed.

Thus, the present invention features novel compounds having the following general formula (I): 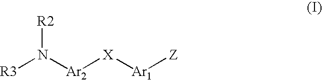
in which:
Ar₁ is an optionally substituted radical of one of the formulae (a)-(e): 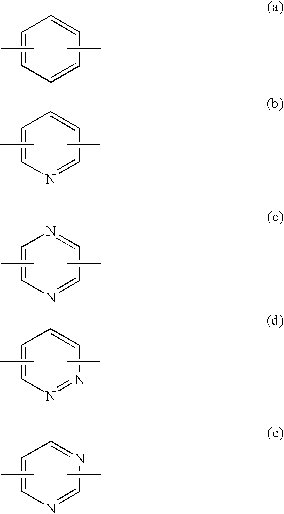
Z is the substituent: 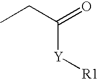
   with the proviso that Z is at the para position with respect to X on the ring Ar₁;
R1 and Y are as defined below;
Ar₂ is an optionally substituted radical of one of the formulae (f)-(n): 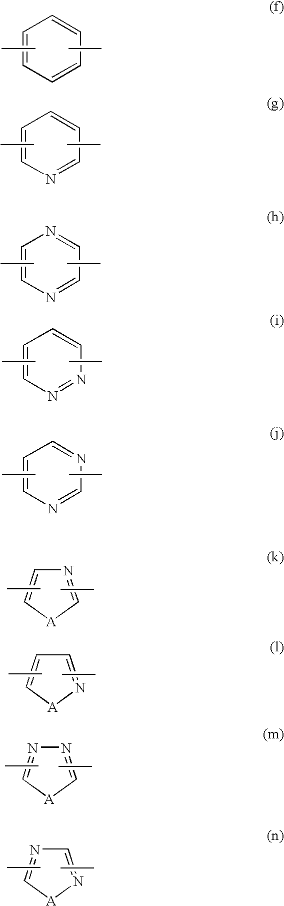
R1 is a hydrogen atom, an alkyl radical having from 1 to 12 carbon atoms, an aryl radical, a heteroaryl radical, an aralkyl radical, a polyether radical, a monohydroxyalkyl radical or a polyhydroxyalkyl radical;
R2 is a hydrogen atom, an alkyl radical having from 1 to 12 carbon atoms, an aryl radical, a heteroaryl radical, an aralkyl radical, a polyether radical, a monohydroxyalkyl radical or a polyhydroxyalkyl radical;
R3 is a hydrogen atom, an alkyl radical having from 1 to 12 carbon atoms, an aryl radical, a heteroaryl radical, an aralkyl radical, a polyether radical, a monohydroxyalkyl radical, a polyhydroxyalkyl radical, a radical COR5 or CSR5;
   R5 is as defined below;
Y is an oxygen or sulfur atom, or the radical N—R4;
   R4 is as defined below;
R4 is a hydrogen atom, an alkyl radical having from 1 to 12 carbon atoms, an aryl radical, a heteroaryl radical, a polyether radical, an aralkyl radical or together forms, with R1 and the nitrogen atom of Y, a heterocycle or a heteroaryl radical;
R5 is an aryl radical, a heteroaryl radical, an aralkyl radical, an alkyl radical having from 1 to 12 carbon atoms, a polyether radical, an alkoxy radical, a monohydroxyalkyl radical, a polyhydroxyalkyl radical, a radical R6-N—R7 or a radical O—R8;
R6, R7 and R8 are as defined below; R6 and R7, which may be identical or different, are each a hydrogen atom, an alkyl radical having from 1 to 12 carbon atoms, a monohydroxyalkyl radical, a polyhydroxyalkyl radical, a polyether radical, an aryl radical, a heteroaryl radical, an aralkyl radical or together form a heterocycle;
R8 is a hydrogen atom, an alkyl radical having from 1 to 12 carbon atoms, a monohydroxyalkyl radical, a polyhydroxyalkyl radical, a polyether radical, an aryl radical, a heteroaryl radical or an aralkyl radical;
X is an S atom, a radical S═O, a radical O═S═O, an Se atom, an O atom, a radical N—R9, a radical C═O, a radical HO—C—R 11 or a radical R10-C—R11;
   R9, R10 and R11 are as defined below;
R9 is a hydrogen atom, a radical —COR12, an alkyl radical having from 1 to 12 carbon atoms, a polyether radical, an aryl radical or an aralkyl radical;
   R12 is as defined below;
R10 and R11, which may be identical or different, are each a hydrogen atom, an alkyl radical having from 1 to 12 carbon atoms, an aryl radical, a heteroaryl radical, an aralkyl radical, a monohydroxyalkyl radical, a polyhydroxyalkyl radical, a polyether radical, an alkoxy radical, or R10 and R11 together form a ring member optionally interrupted by heteroatoms and preferably the rings are dithianyl, dioxanyl, dithiolanyl, dioxolanyl or cyclopropanyl radicals;
A is an S, O or Se atom or a radical N—R13;
R13 is as defined below;
R12 is an alkyl radical having from 1 to 12 carbon atoms;
R13 is a hydrogen atom, an alkyl radical having from 1 to 12 carbon atoms, an aryl radical, a heteroaryl radical, a polyether radical or an aralkyl radical;
   and with the proviso when R2 is a hydrogen atom, R5 is different from an aryl radical and R3 is different from a hydrogen atom,
   and the optical and geometric isomers of the said compounds of formula (I) and salts thereof.

### DETAILED DESCRIPTION OF BEST MODE AND SPECIFIC/PREFERRED EMBODIMENTS OF THE INVENTION

In particular, when the compounds according to the invention are provided in the form of salts, they are salts of an alkali or alkaline-earth metal, zinc salts, or salts of an organic amine.

According to the present invention, the expression “hydroxyl radical” means the —OH radical.

According to the present invention, the expression “alkyl radical having from 1 to 12 carbon atoms” means a hydrogenated or fluorinated, linear or cyclic, optionally branched, radical containing 1 to 12 carbon atoms which may be interrupted by one or more heteroatoms, and preferably the alkyl radicals having from 1 to 12 carbon atoms are methyl, ethyl, isopropyl, butyl, tert-butyl, hexyl, octyl, decyl or cyclohexyl radicals.

The expression “monohydroxyalkyl radical” means a radical having 1 to 6 carbon atoms, and preferably having from 2 to 3 carbon atoms, in particular a 2-hydroxyethyl, 2-hydroxypropyl or 3-hydroxypropyl radical.

The expression “polyhydroxyalkyl radical” means a radical containing from 3 to 6 carbon atoms and from 2 to 5 hydroxyl groups, such as 2,3-dihydroxypropyl, 2,3,4-trihydroxybutyl or 2,3,4,5-tetrahydroxypentyl radicals, or the pentaerythritol residue.

The expression “polyether radical” means a polyether radical having from 1 to 6 carbon atoms interrupted by at least one oxygen atom such as methoxymethoxy, ethoxymethoxy or methoxyethoxymethoxy radicals.

The expression “alkoxy radical having from 1 to 7 carbon atoms” means a radical containing from one to seven carbon atoms such as the methoxy, ethoxy, isopropyloxy, tert-butoxy, hexyloxy, benzyloxy or phenoxy radicals, which may be optionally substituted with an alkyl radical having from 1 to 12 carbon atoms.

The expression “aryl radical” means a phenyl, biphenyl, cinnamyl or naphthyl radical which may be mono- or disubstituted with a halogen atom, a radical CF₃, an alkyl radical having from 1 to 12 carbon atoms, an alkoxy radical having from 1 to 7 carbon atoms, a nitro functional group, a polyether radical, an aryl radical, a benzoyl radical, an alkyl ester group, a carboxylic acid, a hydroxyl radical optionally protected by an acetyl or benzoyl group or an amino functional group optionally protected by an acetyl or benzoyl group or optionally substituted with at least one alkyl having from 1 to 12 carbon atoms.

The expression “aralkyl radical” means a benzyl, phenethyl or naphthalen-2-ylmethyl radical which may be mono- or disubstituted with a halogen atom, a radical CF₃, an alkyl radical having from 1 to 12 carbon atoms, an alkoxy radical having from 1 to 7 carbon atoms, a nitro functional group, a polyether radical, an aryl radical, a benzoyl radical, an alkyl ester group, a carboxylic acid, a hydroxyl radical optionally protected by an acetyl or benzoyl group or an amino functional group optionally protected by an acetyl or benzoyl group or optionally substituted with at least one alkyl having from 1 to 12 carbon atoms.

The expression “heteroaryl radical” is preferably understood to mean an aryl radical interrupted by one or more heteroatoms, such as the pyridyl, furyl, thienyl, isoxazolyl, oxadiazolyl, oxazolyl, isothiazolyl, quinazolinyl, benzothiadiazolyl, benzimidazole, indolyl or benzofuran radical, optionally substituted with at least one halogen, an alkyl having from 1 to 12 carbon atoms, an alkoxy having from 1 to 7 carbon atoms, an aryl radical, a nitro functional group, a polyether radical, a heteroaryl radical, a benzoyl radical, an alkyl ester group, a carboxylic acid, a hydroxyl optionally protected by an acetyl or benzoyl group or an amino functional group optionally protected by an acetyl or benzoyl group or optionally substituted with at least one alkyl having from 1 to 12 carbon atoms.

The expression “heterocycle” is preferably understood to mean the morpholino, piperidino, piperazino, 2-oxopiperidin-1-yl and 2-oxopyrrolidin-1-yl radicals optionally substituted with at least one alkyl group having from 1 to 12 carbon atoms, an alkoxy having from 1 to 7 carbon atoms, an aryl radical, a nitro functional group, a polyether radical, a heteroaryl radical, a benzoyl radical, an alkyl ester group, a carboxylic acid, a hydroxyl optionally protected by an acetyl or benzoyl group or an amino functional group optionally protected by an acetyl or benzoyl group or optionally substituted with at least one alkyl having from 1 to 12 carbon atoms.

Among the compounds of formula (I) falling within the scope of the present invention, the following compounds may be mentioned in particular (alone or as a mixture):
1b. Ethyl[4-(3-aminophenylsulfanyl)phenyl]acetate,
2. Ethyl[4-(4-aminophenylsulfanyl)phenyl]acetate,
3a. Ethyl{4-[3-(3-phenylpropylamino)phenylsulfanyl]phenyl}acetate,
3b. {4-[3-(3-Phenylpropylamino)phenylsulfanyl]phenyl}acetic acid,
4a. Ethyl(4-{3-[bis(3-phenylpropyl)amino]phenylsulfanyl}phenyl)acetate,
4b. (4-{3-[Bis(3-phenylpropyl)amino]phenylsuphanyl}phenyl)acetic acid,
5a. Ethyl[4-(3-Phenethylamino)phenylsulfanylphenyl]acetate,
5b. [4-(3-Phenethylamino)phenylsulfanylphenyl]acetic acid,
6a. Ethyl[4-(3-Diphenethylaminophenylsulfanyl)phenyl]acetate,
6b. [4-(3-Diphenethylaminophenylsulfanyl)phenyl]acetic acid,
7a. Ethyl[4-(3-heptylaminophenylsulfanyl)phenyl]acetate,
7b. [4-(3-Heptylaminophenylsulfanyl)phenyl]acetic acid,
8a. Ethyl[4-(3-diheptylaminophenylsulfanyl)phenyl]acetate,
8b. [4-(3-Diheptylaminophenylsulfanyl)phenyl]acetic acid,
9a. Ethyl[4-(3-butylaminophenylsulfanyl)phenyl]acetate,
9b. [4-(3-Butylaminophenylsulfanyl)phenyl]acetic acid,
10a. Ethyl[4-(3-dibutylaminophenylsulfanyl)phenyl]acetate,
10b. [4-(3-Dibutylaminophenylsulfanyl)phenyl]acetic acid,
11a. Ethyl{4-[4-(3-phenylpropylamino)phenylsulfanyl]phenyl}acetate,
11b. {4-[4-(3-Phenylpropylamino)phenylsulfanyl]phenyl}acetic acid,
12a. Ethyl(4-{4-[bis(3-phenylpropyl)amino]phenylsulfanyl}phenyl)acetate,
12b. (4-{4-[Bis(3-phenylpropyl)amino]phenylsulfanyl}phenyl)acetic acid,
13a. Ethyl[4-(4-phenethylaminophenylsulfanyl)phenyl]acetate,
13b. [4-(4-Phenethylaminophenylsulfanyl)phenyl]acetic acid,
14a. Ethyl[4-(4-diphenethylaminophenylsulfanyl)phenyl]acetate,
14b. [4-(4-Diphenethylaminophenylsulfanyl)phenyl]acetic acid,
15a. Ethyl[4-(4-heptylaminophenylsulfanyl)phenyl]acetate,
15b. [4-(4-Heptylaminophenylsulfanyl)phenyl]acetic acid,
16a. Ethyl[4-(4-diheptylaminophenylsulfanyl)phenyl]acetate,
16b. [4-(4-Diheptylaminophenylsulfanyl)phenyl]acetic acid,
17a. Ethyl[4-(4-dibutylaminophenylsulfanyl)phenyl]acetate,
17b. [4-(4-Dibutylaminophenylsulfanyl)phenyl]acetic acid,
18a. Ethyl(4-{3-[3-benzyl-1-(3-phenylpropyl)ureido]-phenylsulfanyl}phenyl)acetate,
18b. (4-{3-[3-Benzyl-1-(3-phenylpropyl)ureido]phenylsulfanyl}phenyl)acetic acid,
19a. Ethyl(4-{3-[3-phenyl-1-(3-phenylpropyl)ureido]phenylsulfanyl}phenyl)acetate,
19b. (4-{3-[3-Phenyl-1-(3-phenylpropyl)ureido]phenylsulfanyl}phenyl)acetic acid,
20a. Ethyl(4-{3-[3-(2,3-dichlorophenyl)-1-(3-phenylpropyl)ureido]phenylsulfanyl}phenyl)acetate,
20b. (4-{3-[3-(2,3-Dichlorophenyl)-1-(3-phenylpropyl)ureido]phenylsulfanyl}phenyl)acetic acid,
21a. Ethyl(4-{3-[3-heptyl-1-(3-phenylpropyl)ureido]phenylsulfanyl}phenyl)acetate,
21 b. (4-{3-[3-Heptyl-1-(3-phenylpropyl)ureido]phenylsulfanyl}phenyl)acetic acid,
22a. Ethyl(4-{3-[3-phenethyl-1-(3-phenylpropyl)ureido]phenylsulfanyl}phenyl)acetate,
22b. (4-{3-[3-Phenethyl-1-(3-phenylpropyl)ureido]phenylsulfanyl}phenyl)acetic acid,
23a. Ethyl(4-{3-[1-(3-phenylpropyl)-3-(4-trifluoromethylphenyl)ureido]phenylsulfanyl}phenyl)acetate,
23b. (4-{3-[1-(3-Phenylpropyl)-3-(4-trifluoromethylphenyl)ureido]phenylsulfanyl}phenyl)acetic acid,
24a. Ethyl(4-{3-[3-(4-methoxyphenyl)-1-(3-phenylpropyl)ureido]phenylsulfanyl}phenyl)acetate,
24b. (4-{3-[3-(4-Methoxyphenyl)-1-(3-phenylpropyl)ureido]phenylsulfanyl}phenyl)acetic acid,
25a. Ethyl(4-{3-[3-adamantan-1-yl-1-(3-phenylpropyl)-ureido]phenylsulfanyl}phenyl)acetate,
25b. (4-{3-[3-Adamantan-1-yl-1-(3-phenylpropyl)ureido]-phenylsulfanyl}phenyl)acetic acid,
26a. Ethyl(4-{3-[3-(2-phenoxyphenyl)-1-(3-phenylpropyl)-ureido]phenylsulfanyl}phenyl)acetate,
26b. (4-{3-[3-(2-Phenoxyphenyl)-1-(3-phenylpropyl)-ureido]phenylsulfanyl}phenyl)acetic acid,
27a. Ethyl(4-{3-[3-allyl-1-(3-phenylpropyl)ureido]-phenylsulfanyl}phenyl)acetate,
27b. (4-{3-[3-Allyl-1-(3-phenylpropyl)ureido]phenylsulfanyl}phenyl)acetic acid,
28a. Ethyl(4-{3-[3-cyclohexyl-1-(3-phenylpropyl)ureido]-phenylsulfanyl}phenyl)acetate,
28b. (4-{3-[3-Cyclohexyl-1-(3-phenylpropyl)ureido]-phenylsulfanyl}phenyl)acetic acid,
29a. Ethyl(4-{3-[3-(2-nitrophenyl)-1-(3-phenylpropyl)-ureido]phenylsulfanyl}phenyl)acetate,
29b. (4-{3-[3-(2-Nitrophenyl)-1-(3-phenylpropyl)ureido]-phenylsulfanyl}phenyl)acetic acid,
30a. Ethyl(4-{3-[3-hexyl-1-(3-phenylpropyl)ureido]-phenylsulfanyl}phenyl)acetate,
30b. (4-{3-[3-Hexyl-1-(3-phenylpropyl)ureido]phenylsulfanyl}phenyl)acetic acid,
31a. Ethyl(4-{3-[3-naphthalen-2-yl-1-(3-phenylpropyl)ureido]-phenylsulfanyl}phenyl)acetate,
31 b. (4-{3-[3-Naphthalen-2-yl-1-(3-phenylpropyl)ureido]-phenylsulfanyl}phenyl)acetic acid,
32a. Ethyl(4-{3-[3-(2-ethoxyphenyl)-1-(3-phenylpropyl)-ureido]phenylsulfanyl}phenyl)acetate,
32b. (4-{3-[3-(2-Ethoxyphenyl)-1-(3-phenylpropyl)ureido]-phenylsulfanyl}phenyl)acetic acid,
33a. Ethyl(4-{3-[3-(4-butoxyphenyl)-1-(3-phenylpropyl)-ureido]phenylsulfanyl}phenyl)acetate,
33b. (4-{3-[3-(4-Butoxyphenyl)-1-(3-phenylpropyl)-ureido]phenylsulfanyl}phenyl)acetic acid,
34a. Ethyl(4-{3-[3-pentyl-1-(3-phenylpropyl)ureido]-phenylsulfanyl}phenyl)acetate,
34b. (4-{3-[3-Pentyl-1-(3-phenylpropyl)ureido]phenylsulfanyl}phenyl)acetic acid,
35a. Ethyl(4-{3-[3-butyl-1-(3-phenylpropyl)ureido]-phenylsulfanyl}phenyl)acetate,
35b. (4-{3-[3-Butyl-1-(3-phenylpropyl)ureido]phenylsulfanyl}phenyl)acetic acid,
36a. Ethyl(4-{3-[3-(4-dimethylaminophenyl)-1-(3-phenylpropyl)ureido]phenylsulfanyl}phenyl)acetate,
36b. (4-{3-[3-(4-Dimethylaminophenyl)-1-(3-phenylpropyl)-ureido]phenylsulfanyl}phenyl)acetic acid,
37a. Ethyl{4-[3-(3-benzyl-1-phenethylureido)phenylsulfanyl]-phenyl}acetate,
37b. {4-[3-(3-Benzyl-1-phenethylureido)phenylsulfanyl]phenyl}acetic acid,
38a. Ethyl{4-[3-(1-phenethyl-3-phenylureido)phenylsulfanyl]-phenyl}acetate,
38b. {4-[3-(1-Phenethyl-3-phenylureido)phenylsulfanyl]phenyl}acetic acid,
39a. Ethyl(4-{3-[3-(2,3-dichlorophenyl)-1-phenethylureido]-phenylsulfanyl}phenyl)acetate,
39b. (4-{3-[3-(2,3-Dichlorophenyl)-1-phenethylureido]-phenylsulfanyl}phenyl)acetic acid,
40a. Ethyl{4-[3-(3-heptyl-1-phenethylureido)phenylsulfanyl]phenyl}acetate,
40b. {4-[3-(3-Heptyl-1-phenethylureido)phenylsulfanyl]phenyl}acetic acid,
41a. Ethyl{4-[3-(1,3-diphenethylureido)phenylsulfanyl]phenyl}acetate,
41b. {4-[3-(1,3-Diphenethylureido)phenylsulfanyl]phenyl}acetic acid,
42a. Ethyl(4-{3-[1-phenethyl-3-(4-trifluoromethyl-phenyl)ureido]phenylsulfanyl}phenyl)acetate,
42b. (4-{3-[1-Phenethyl-3-(4-trifluoromethylphenyl)-ureido]phenylsulfanyl}phenyl)acetic acid,
43a. Ethyl(4-{3-[3-(4-methoxyphenyl)-1-phenethylureido]phenylsulfanyl}phenyl)acetate,
43b. (4-{3-[3-(4-Methoxyphenyl)-1-phenethylureido]-phenylsulfanyl}phenyl)acetic acid,
44a. Ethyl{4-[3-(3-adamantan-1-yl-1-phenethylureido)-phenylsulfanyl]phenyl}acetate,
44b. {4-[3-(3-Adamantan-1-yl-1-phenethylureido)phenyl-sulfanyl]phenyl}acetic acid,
45a. Ethyl(4-{3-[1-phenethyl-3-(2-phenoxyphenyl)-ureido]phenylsulfanyl}phenyl)acetate,
45b. (4-{3-[1-Phenethyl-3-(2-phenoxyphenyl)ureido]-phenylsulfanyl}phenyl)acetic acid,
46a. Ethyl{4-[3-(3-allyl-1-phenethylureido)phenylsulfanyl]phenyl}acetate,
46b. {4-[3-(3-Ally-1-phenethylureido)phenylsulfanyl]phenyl}acetic acid,
47a. Ethyl{4-[3-(3-cyclohexyl-1-phenethylureido)-phenylsulfanyl]phenyl}acetate,
47b. {4-[3-(3-Cyclohexyl-1-phenethylureido)phenylsulfanyl]phenyl}acetic acid,
48a. Ethyl(4-{3-[3-(2-nitrophenyl)-1-phenethylureido]-phenylsulfanyl}phenyl)acetate,
48b. (4-{3-[3-(2-Nitrophenyl)-1-phenethylureido]phenyl-sulfanyl}phenyl)acetic acid,
49a. Ethyl{4-[3-(3-hexyl-1-phenethylureido)phenylsulfanyl]phenyl}acetate,
49b. {4-[3-(3-Hexyl-1-phenethylureido)phenylsulfanyl]phenyl}acetic acid,
50a. Ethyl{4-[3-(3-naphthalen-2-yl-1-phenethylureido)-phenylsulfanyl]phenyl}acetate,
50b. {4-[3-(3-Naphthalen-2-yl-1-phenethylureido)phenyl-sulfanyl]phenyl}acetic acid,
51a. Ethyl(4-{3-[3-(2-ethoxyphenyl)-1-phenethylureido]phenylsulfanyl}phenyl)acetate,
51b. (4-{3-[3-(2-Ethoxyphenyl)-1-phenethylureido]-phenylsulfanyl}phenyl)acetic acid,
52a. Ethyl(4-{3-[3-(4-butoxyphenyl)-1-phenethylureido]phenylsulfanyl}phenyl)acetate,
52b. (4-{3-[3-(4-Butoxyphenyl)-1-phenethylureido]-phenylsulfanyl}phenyl)acetic acid,
53a. Ethyl{4-[3-(3-pentyl-1-phenethylureido)phenylsulfanyl]phenyl}acetate,
53b. {4-[3-(3-Pentyl-1-phenethylureido)phenylsulfanyl]phenyl}acetic acid,
54a. Ethyl{4-[3-(3-butyl-1-phenethylureido)phenylsulfanyl]phenyl}acetate,
54b. {4-[3-(3-Butyl-1-phenethylureido)phenylsulfanyl]phenyl}acetic acid,
55a. Ethyl(4-{3-[3-(4-dimethylaminophenyl)-1-phenethylureido]phenylsulfanyl}phenyl)acetate,
55b. (4-{3-[3-(4-Dimethylaminophenyl)-1-phenethylureido]phenylsulfanyl}phenyl)acetic acid,
56a. Ethyl{4-[3-(3-benzyl-1-heptylureido)phenylsulfanyl]phenyl}acetate,
56b. {4-[3-(3-Benzyl-1-heptylureido)phenylsulfanyl]phenyl}acetic acid,
57a. Ethyl{4-[3-(1-heptyl-3-phenylureido)phenylsulfanyl]phenyl}acetate,
57b. {4-[3-(1-Heptyl-3-phenylureido)phenylsulfanyl]phenyl}acetic acid,
58a. Ethyl{4-[3-(1,3-diheptylureido)phenylsulfanyl]phenyl}acetate,
58b. {4-[3-(1,3-Diheptylureido)phenylsulfanyl]phenyl}acetic acid,
59a. Ethyl{4-[3-(1-heptyl-3-phenethylureido)phenylsulfanyl]phenyl}acetate,
59b. {4-[3-(1-Heptyl-3-phenethylureido)phenylsulfanyl]phenyl}acetic acid,
60a. Ethyl(4-{3-[1-heptyl-3-(4-trifluoromethylphenyl)-ureido]phenylsulfanyl}phenyl)acetate,
60b. (4-{3-[1-Heptyl-3-(4-trifluoromethylphenyl)-ureido]phenylsulfanyl}phenyl)acetic acid,
61a. Ethyl(4-{3-[1-heptyl-3-(4-methoxyphenyl)ureido]-phenylsulfanyl}phenyl)acetate,
61 b. (4-{3-[1-Heptyl-3-(4-methoxyphenyl)ureido]-phenylsulfanyl}phenyl)acetic acid,
62a. Ethyl{4-[3-(3-adamantan-1-yl-1-heptylureido)-phenylsulfanyl]phenyl}acetate,
62b. {4-[3-(3-Adamantan-1-yl-1-heptylureido)phenylsulfanyl]phenyl}acetic acid,
63a. Ethyl(4-{3-[1-heptyl-3-(2-phenoxyphenyl)ureido]-phenylsulfanyl}phenyl)acetate,
63b. (4-{3-[1-Heptyl-3-(2-phenoxyphenyl)ureido]-phenylsulfanyl}phenyl)acetic acid,
64a. Ethyl{4-[3-(3-allyl-1-heptylureido)phenylsulfanyl]phenyl}acetate,
64b. {4-[3-(3-Allyl-1-heptylureido)phenylsulfanyl]phenyl}acetic acid,
65a. Ethyl{4-[3-(3-cyclohexyl-1-heptylureido)phenyl-sulfanyl]phenyl}acetate,
65b. {4-[3-(3-Cyclohexyl-1-heptylureido)phenylsulfanyl]phenyl}acetic acid,
66a. Ethyl(4-{3-[1-heptyl-3-(2-nitrophenyl)ureido]-phenylsulfanyl}phenyl)acetate,
66b. (4-{3-[1-Heptyl-3-(2-nitrophenyl)ureido]phenylsulfanyl}phenyl)acetic acid,
67a. Ethyl{4-[3-(1-heptyl-3-hexylureido)phenylsulfanyl]phenyl}acetate,
67b. {4-[3-(1-Heptyl-3-hexylureido)phenylsulfanyl]phenyl}acetic acid,
68a. Ethyl{4-[3-(1-heptyl-3-naphthalen-2-ylureido)-phenylsulfanyl]phenyl}acetate,
68b. {4-[3-(1-Heptyl-3-naphthalen-2-ylureido)phenylsulfanyl]phenyl}acetic acid,
69a. Ethyl(4-{3-[3-(2-ethoxyphenyl)-1-heptylureido]-phenylsulfanyl}phenyl)acetate,
69b. (4-{3-[3-(2-Ethoxyphenyl)-1-heptylureido]phenylsulfanyl}phenyl)acetic acid,
70a. Ethyl(4-{3-[3-(4-butoxyphenyl)-1-heptylureido]-phenylsulfanyl}phenyl)acetate,
70b. (4-{3-[3-(4-Butoxyphenyl)-1-heptylureido]phenylsulfanyl}phenyl)acetic acid,
71a. Ethyl{4-[3-(1-heptyl-3-pentylureido)phenylsulfanyl]phenyl}acetate,
71b. {4-[3-(1-Heptyl-3-pentylureido)phenylsulfanyl]phenyl}acetic acid,
72a. Ethyl{4-[3-(3-butyl-1-heptylureido)phenylsulfanyl]phenyl}acetate,
72b. {4-[3-(3-Butyl-1-heptylureido)phenylsulfanyl]phenyl}acetic acid,
73a. Ethyl(4-{3-[3-(4-dimethylaminophenyl)-1-heptyl-ureido]phenylsulfanyl}phenyl)acetate,
73b. (4-{3-[3-(4-Dimethylaminophenyl)-1-heptylureido]-phenylsulfanyl}phenyl)acetic acid,
74a. Ethyl{4-[3-(3-benzyl-1-butylureido)phenylsulfanyl]phenyl}acetate,
74b. {4-[3-(3-Benzyl-1-butylureido)phenylsulfanyl]phenyl}acetic acid,
75a. Ethyl{4-[3-(1-butyl-3-phenylureido)phenylsulfanyl]phenyl}acetate,
75b. {4-[3-(1-Butyl-3-phenylureido)phenylsulfanyl]phenyl}acetic acid,
76a. Ethyl(4-{3-[1-butyl-3-(2,3-dichlorophenyl)-ureido]phenylsulfanyl}phenyl)acetate,
76b. (4-{3-[1-Butyl-3-(2,3-dichlorophenyl)ureido]-phenylsulfanyl}phenyl)acetic acid,
77a. Ethyl{4-[3-(1-butyl-3-heptylureido)phenylsulfanyl]phenyl}acetate,
77b. {4-[3-(1-Butyl-3-heptylureido)phenylsulfanyl]phenyl}acetic acid,
78a. Ethyl{4-[3-(1-butyl-3-phenethylureido)phenylsulfanyl]phenyl}acetate,
78b. {4-[3-(1-Butyl-3-phenethylureido)phenylsulfanyl]phenyl}acetic acid,
79a. Ethyl(4-{3-[1-butyl-3-(4-trifluoromethylphenyl)-ureido]phenylsulfanyl}phenyl)acetate,
79b. (4-{3-[1-Butyl-3-(4-trifluoromethylphenyl)ureido]-phenylsulfanyl}phenyl)acetic acid,
80a. Ethyl(4-{3-[1-butyl-3-(4-methoxyphenyl)ureido]-phenylsulfanyl}phenyl)acetate,
80b. (4-{3-[1-Butyl-3-(4-methoxyphenyl)ureido]phenylsulfanyl}phenyl)acetic acid,
81a. Ethyl{4-[3-(3-adamantan-1-yl-1-butylureido)-phenylsulfanyl]phenyl}acetate,
81 b. {4-[3-(3-Adamantan-1-yl-1-butylureido)phenylsulfanyl]phenyl}acetic acid,
82a. Ethyl(4-{3-[1-butyl-3-(2-phenoxyphenyl)ureido]-phenylsulfanyl}phenyl)acetate,
82b. (4-{3-[1-Butyl-3-(2-phenoxyphenyl)ureido]phenylsulfanyl}phenyl)acetic acid,
83a. Ethyl{4-[3-(3-allyl-1-butylureido)phenylsulfanyl]phenyl}acetate,
83b. {4-[3-(3-Allyl-1-butylureido)phenylsulfanyl]phenyl}acetic acid,
84a. Ethyl{4-[3-(1-butyl-3-cyclohexylureido)phenylsulfanyl]phenyl}acetate,
84b. {4-[3-(1-Butyl-3-cyclohexylureido)phenylsulfanyl]phenyl}acetic acid,
85a. Ethyl(4-{3-[1-butyl-3-(2-nitrophenyl)ureido]-phenylsulfanyl}phenyl)acetate,
85b. (4-{3-[1-Butyl-3-(2-nitrophenyl)ureido]phenylsulfanyl}phenyl)acetic acid,
86a. Ethyl{4-[3-(1-butyl-3-hexylureido)phenylsulfanyl]phenyl}acetate,
86b. {4-[3-(1-Butyl-3-hexylureido)phenylsulfanyl]phenyl}acetic acid,
87a. Ethyl{4-[3-(1-butyl-3-naphthalen-2-ylureido)-phenylsulfanyl]phenyl}acetate,
87b. {4-[3-(1-Butyl-3-naphthalen-2-ylureido)phenylsulfanyl]phenyl}acetic acid,
88a. Ethyl(4-{3-[1-butyl-3-(2-ethoxyphenyl)ureido]-phenylsulfanyl}phenyl)acetate,
88b. (4-{3-[1-Butyl-3-(2-ethoxyphenyl)ureido]phenylsulfanyl}phenyl)acetic acid,
89a. Ethyl(4-{3-[3-(4-butoxyphenyl)-1-butylureido]-phenylsulfanyl}phenyl)acetate,
89b. (4-{3-[3-(4-Butoxyphenyl)-1-butylureido]phenylsulfanyl}phenyl)acetic acid,
90a. Ethyl{4-[3-(1-butyl-3-pentylureido)phenylsulfanyl]phenyl}acetate,
90b. {4-[3-(1-Butyl-3-pentylureido)phenylsulfanyl]phenyl}acetic acid,
91a. Ethyl{4-[3-(1,3-dibutylureido)phenylsulfanyl]phenyl}acetate,
91 b. {4-[3-(1,3-Dibutylureido)phenylsulfanyl]phenyl}acetic acid,
92a. Ethyl(4-{4-[3-benzyl-1-(3-phenylpropyl)ureido]-phenylsulfanyl}phenyl)acetate,
92b. (4-{4-[3-Benzyl-1-(3-phenylpropyl)ureido]phenylsulfanyl}phenyl)acetic acid,
93a. Ethyl(4-{4-[3-heptyl-1-(3-phenylpropyl)ureido]-phenylsulfanyl}phenyl)acetate,
93b. (4-{4-[3-Heptyl-1-(3-phenylpropyl)ureido]phenylsulfanyl}phenyl)acetic acid,
94a. Ethyl(4-{4-[3-phenethyl-1-(3-phenylpropyl)-ureido]phenylsulfanyl}phenyl)acetate,
94b. (4-{4-[3-Phenethyl-1-(3-phenylpropyl)ureido]-phenylsulfanyl}phenyl)acetic acid,
95a. Ethyl(4-{4-[1-(3-phenylpropyl)-3-(4-tri-fluoromethylphenyl)ureido]phenylsulfanyl}phenyl)acetate,
95b. (4-{4-[1-(3-Phenylpropyl)-3-(4-tri-fluoromethylphenyl)ureido]phenylsulfanyl}phenyl)acetic acid,
96a. Ethyl(4-{4-[3-(2-phenoxyphenyl)-1-(3-phenyl-propyl)ureido]phenylsulfanyl}phenyl)acetate,
96b. (4-{4-[3-(2-Phenoxyphenyl)-1-(3-phenyl-propyl)ureido]phenylsulfanyl}phenyl)acetic acid,
97a. Ethyl(4-{4-[3-allyl-1-(3-phenylpropyl)ureido]-phenylsulfanyl}phenyl)acetate,
97b. (4-{4-[3-Allyl-1-(3-phenylpropyl)ureido]phenylsulfanyl}phenyl)acetic acid,
98a. Ethyl(4-{4-[3-cyclohexyl-1-(3-phenylpropyl)-ureido]phenylsulfanyl}phenyl)acetate,
98b. (4-{4-[3-Cyclohexyl-1-(3-phenylpropyl)ureido]-phenylsulfanyl}phenyl)acetic acid,
99a. Ethyl(4-{4-[3-(2-nitrophenyl)-1-(3-phenyl-propyl)ureido]phenylsulfanyl}phenyl)acetate,
99b. (4-{4-[3-(2-Nitrophenyl)-1-(3-phenylpropyl)-ureido]phenylsulfanyl}phenyl)acetic acid,
100a. Ethyl(4-{4-[3-hexyl-1-(3-phenylpropyl)-ureido]phenylsulfanyl}phenyl)acetate,
100b. (4-{4-[3-Hexyl-1-(3-phenylpropyl)ureido]phenylsulfanyl}phenyl)acetic acid,
101a. Ethyl(4-{4-[3-naphthalen-2-yl-1-(3-phenyl-propyl)ureido]phenylsulfanyl}phenyl)acetate,
101 b. (4-{4-[3-Naphthalen-2-yl-1-(3-phenyl-propyl)ureido]phenylsulfanyl}phenyl)acetic acid,
102a. Ethyl(4-{4-[3-(2-ethoxyphenyl)-1-(3-phenyl-propyl)ureido]phenylsulfanyl}phenyl)acetate,
102b. (4-{4-[3-(2-Ethoxyphenyl)-1-(3-phenylpropyl)-ureido]phenylsulfanyl}phenyl)acetic acid,
103a. Ethyl(4-{4-[3-(4-butoxyphenyl)-1-(3-phenyl-propyl)ureido]phenylsulfanyl}phenyl)acetate,
103b. (4-{4-[3-(4-Butoxyphenyl)-1-(3-phenylpropyl)-ureido]phenylsulfanyl}phenyl)acetic acid,
104a. Ethyl(4-{4-[3-pentyl-1-(3-phenylpropyl)-ureido]phenylsulfanyl}phenyl)acetate,
104b. (4-{4-[3-Pentyl-1-(3-phenylpropyl)ureido]-phenylsulfanyl}phenyl)acetic acid,
105a. Ethyl(4-{4-[3-butyl-1-(3-phenylpropyl)ureido]-phenylsulfanyl}phenyl)acetate,
105b. (4-{4-[3-Butyl-1-(3-phenylpropyl)ureido]phenylsulfanyl}phenyl)acetic acid,
106a. Ethyl{4-[4-(3-benzyl-1-phenethylureido)-phenylsulfanyl]phenyl}acetate,
106b. {4-[4-(3-Benzyl-1-phenethylureido)phenylsulfanyl]phenyl}acetic acid,
107a. Ethyl{4-[4-(1-phenethyl-3-phenylureido)phenyl-sulfanyl]phenyl}acetate,
107b. {4-[4-(1-Phenethyl-3-phenylureido)phenylsulfanyl]phenyl}acetic acid.
108a. Ethyl(4-{4-[3-(2,3-dichlorophenyl)-1-phenethylureido]phenylsulfanyl}phenyl)acetate,
108b. (4-{4-[3-(2,3-Dichlorophenyl)-1-phenethylureido]-phenylsulfanyl}phenyl)acetic acid,
109a. Ethyl{4-[4-(3-heptyl-1-phenethylureido)phenyl-sulfanyl]phenyl}acetate,
109b. {4-[4-(3-Heptyl-1-phenethylureido)phenylsulfanyl]phenyl}acetic acid,
110a. Ethyl{4-[4-(1,3-diphenethylureido)phenylsulfanyl]phenyl}acetate,
110b. {4-[4-(1,3-Diphenethylureido)phenylsulfanyl]phenyl}acetic acid,
111a. Ethyl(4-{4-[1-phenethyl-3-(4-trifluoro-methylphenyl)ureido]phenylsulfanyl}phenyl)acetate,
111 b. (4-{4-[1-Phenethyl-3-(4-trifluoromethyl-phenyl)ureido]phenylsulfanyl}phenyl)acetic acid,
112a. Ethyl(4-{4-[3-(4-methoxyphenyl)-1-phenethylureido]phenylsulfanyl}phenyl)acetate,
112b. (4-{4-[3-(4-Methoxyphenyl)-1-phenethylureido]-phenylsulfanyl}phenyl)acetic acid,
113a. Ethyl{4-[4-(3-adamantan-1-yl-1-phenethyl-ureido)phenylsulfanyl]phenyl}acetate,
113b. {4-[4-(3-Adamantan-1-yl-1-phenethylureido)-phenylsulfanyl]phenyl}acetic acid,
114a. Ethyl(4-{4-[1-phenethyl-3-(2-phenoxyphenyl)-ureido]phenylsulfanyl}phenyl)acetate,
114b. (4-{4-[1-Phenethyl-3-(2-phenoxyphenyl)ureido]-phenylsulfanyl}phenyl)acetic acid,
115a. Ethyl{4-[4-(3-allyl-phenethylureido)phenylsulfanyl]phenyl}acetate,
115b. {4-[4-(3-Allyl-1-phenethylureido)phenylsulfanyl]phenyl}acetic acid,
116a. Ethyl{4-[4-(3-cyclohexyl-1-phenethylureido)-phenylsulfanyl]phenyl}acetate,
116b. {4-[4-(3-Cyclohexyl-1-phenethylureido)phenylsulfanyl]phenyl}acetic acid,
117a. Ethyl(4-{4-[3-(2-nitrophenyl)-1-phenethyl-ureido]phenylsulfanyl}phenyl)acetate,
117b. (4-{4-[3-(2-Nitrophenyl)-1-phenethylureido]-phenylsulfanyl}phenyl)acetic acid,
118a. Ethyl{4-[4-(3-hexyl-1-phenethylureido)-phenylsulfanyl]phenyl}acetate,
118b. {4-[4-(3-Hexyl-1-phenethylureido)phenylsulfanyl]phenyl}acetic acid,
119a. Ethyl{4-[4-(3-naphthalen-2-yl-1-phenethyl-ureido)phenylsulfanyl]phenyl}acetate,
119b. {4-[4-(3-Naphthalen-2-yl-1-phenethylureido)-phenylsulfanyl]phenyl}acetic acid,
120a. Ethyl(4-{4-[3-(2-ethoxyphenyl)-1-phenethylureido]phenylsulfanyl}phenyl)acetate,
120b. (4-{4-[3-(2-Ethoxyphenyl)-1-phenethylureido]-phenylsulfanyl}phenyl)acetic acid,
121a. Ethyl(4-{4-[3-(4-butoxyphenyl)-1-phenethylureido]phenylsulfanyl}phenyl)acetate,
121 b. (4-{4-[3-(4-Butoxyphenyl)-1-phenethylureido]-phenylsulfanyl}phenyl)acetic acid,
122a. Ethyl{4-[4-(3-pentyl-1-phenethylureido)phenyl-sulfanyl]phenyl}acetate,
122b. {4-[4-(3-Pentyl-1-phenethylureido)phenylsulfanyl]phenyl}acetic acid,
123a. Ethyl{4-[4-(3-butyl-1-phenethylureido)phenylsulfanyl]phenyl}acetate,
123b. {4-[4-(3-Butyl-1-phenethylureido)phenylsulfanyl]phenyl}acetic acid,
124a. Ethyl{4-[4-(3-benzyl-1-heptylureido)phenylsulfanyl]phenyl}acetate,
124b. {4-[4-(3-Benzyl-1-heptylureido)phenylsulfanyl]phenyl}acetic acid,
125a. Ethyl{4-[4-(1-heptyl-3-phenylureido)phenylsulfanyl]phenyl}acetate,
125b. {4-[4-(1-Heptyl-3-phenylureido)phenylsulfanyl]phenyl}acetic acid,
126a. Ethyl(4-{4-[3-(2,3-dichlorophenyl)-1-heptyl-ureido]phenylsulfanyl}phenyl)acetate,
126b. (4-{4-[3-(2,3-Dichlorophenyl)-1-heptylureido]-phenylsulfanyl}phenyl)acetic acid,
127a. Ethyl{4-[4-(1,3-diheptylureido)phenylsulfanyl]phenyl}acetate,
127b. {4-[4-(1,3-Diheptylureido)phenylsulfanyl]phenyl}acetic acid,
128a. Ethyl{4-[4-(1-heptyl-3-phenethylureido)phenyl-sulfanyl]phenyl}acetate,
128b. {4-[4-(1-Heptyl-3-phenethylureido)phenylsulfanyl]phenyl}acetic acid,
129a. Ethyl(4-{4-[1-heptyl-3-(4-trifluoro-methylphenyl)ureido]phenylsulfanyl}phenyl)acetate,
129b. (4-{4-[1-Heptyl-3-(4-trifluoromethyl-phenyl)ureido]phenylsulfanyl}phenyl)acetic acid,
130a. Ethyl(4-{4-[1-heptyl-3-(4-methoxyphenyl)ureido]-phenylsulfanyl}phenyl)acetate,
130b. (4-{4-[1-Heptyl-3-(4-methoxyphenyl)ureido]phenyl-sulfanyl}phenyl)acetic acid,
131a. Ethyl{4-[4-(3-adamantan-1-yl-1-heptylureido)-phenylsulfanyl]phenyl}acetate,
131 b. {4-[4-(3-Adamantan-1-yl-1-heptylureido)phenylsulfanyl]phenyl}acetic acid,
132a. Ethyl(4-{4-[1-heptyl-3-(2-phenoxyphenyl)-ureido]phenylsulfanyl}phenyl)acetate,
132b. (4-{4-[1-Heptyl-3-(2-phenoxyphenyl)ureido]-phenylsulfanyl}phenyl)acetic acid,
133a. Ethyl{4-[4-(3-allyl-1-heptylureido)phenylsulfanyl]phenyl}acetate,
133b. {4-[4-(3-Allyl-1-heptylureido)phenylsulfanyl]phenyl}acetic acid,
134a. Ethyl{4-[4-(3-cyclohexyl-1-heptylureido)phenyl-sulfanyl]phenyl}acetate,
134b. {4-[4-(3-Cyclohexyl-1-heptylureido)phenylsulfanyl]phenyl}acetic acid,
135a. Ethyl(4-{4-[1-heptyl-3-(2-nitrophenyl)ureido]-phenylsulfanyl}phenyl)acetate,
135b. (4-{4-[1-Heptyl-3-(2-nitrophenyl)ureido]phenylsulfanyl}phenyl)acetic acid,
136a. Ethyl{4-[4-(1-heptyl-3-hexylureido)phenylsulfanyl]phenyl}acetate,
136b. {4-[4-(1-Heptyl-3-hexylureido)phenylsulfanyl]phenyl}acetic acid,
137a. Ethyl{4-[4-(1-heptyl-3-naphthalen-2-ylureido)-phenylsulfanyl]phenyl}acetate,
137b. {4-[4-(1-Heptyl-3-naphthalen-2-ylureido)phenylsulfanyl]phenyl}acetic acid,
138a. Ethyl(4-{4-[3-(2-ethoxyphenyl)-1-heptylureido]-phenylsulfanyl}phenyl)acetate,
138b. (4-{4-[3-(2-Ethoxyphenyl)-1-heptylureido]-phenylsulfanyl}phenyl)acetic acid,
139a. Ethyl(4-{4-[3-(4-butoxyphenyl)-1-heptylureido]-phenylsulfanyl}phenyl)acetate,
139b. (4-{4-[3-(4-Butoxyphenyl)-1-heptylureido]phenyl-sulfanyl}phenyl)acetic acid,
140a. Ethyl{4-[4-(1-heptyl-3-pentylureido)phenylsulfanyl]phenyl}acetate,
140b. {4-[4-(1-Heptyl-3-pentylureido)phenylsulfanyl]phenyl}acetic acid,
141a. Ethyl{4-[4-(3-butyl-1-heptylureido)phenylsulfanyl]phenyl}acetate,
141b. {4-[4-(3-Butyl-1-heptylureido)phenylsulfanyl]phenyl}acetic acid.

A general description of the preparation of the compounds of general formula of the appended Figure of Drawing is given below.

The reaction scheme described in Figure of Drawing is a general scheme allowing the production of the compounds according to the invention.

The compounds of general formula (I) may be obtained (Figure of Drawing) by coupling a thiol, an alcohol, an amine or a seleniated derivate (depend on X value) with an aromatic iodinated compound, using a metal catalyst such as nickel or palladium derivatives, in the presence of a hydride donor such as sodium borohydride and if necessary a base. Concerning diaryl amine compounds, the copper or palladium catalyzed amination (Tetrahedron 58, (2002) 2041-2075) of the nitro aniline compound with aryl halogenide may be employed, followed by the reduction of the nitro to the corresponding amino group. Concerning the preparation of diaryl ether, coupling of the corresponding alkoxide catalyzed by palladium may be employed. Concerning the preparation of diaryl ketone compounds, palladium catalyzed conversion of halogenoaryl derivatives compound to the corresponding organotin derivatives followed by a palladium catalysed coupling with acyl chloride derivative may afford the target product. The ketone might be protected in order to avoid problems during reductive amination. The next step is a reductive amination of the preceding amine and of an aldehyde, which may be carried out with isolation of the intermediate imine or otherwise, followed by reduction of the latter by the action of a reducing agent such as NaBH₃CN. The alkylated amine obtained can then be subjected to the action of an isocyanate or an isothiocyanate in a solvent such as dichloromethane to give the corresponding urea or thiourea. It can also be further alkylated by reductive amination reaction in the presence of an aldehyde under the same conditions as above. The amide may also be formed by the action of an acid in the presence of a coupling agent such as O-(7-azabenzotriazol-1-yl)-N,N,N′,N′-tetramethyluronium hexafluorophosphate (HATU) in the presence of a base such as DIEA or an acyl halide and a base. The derivatives obtained are then saponified by the action, for example, of a base such as NaOH to give the corresponding acids. The sulfated compounds (X: S) oxydated by the action of metachloroperbenzoic acid (MCPBA) in the presence of dichloromethane.

The compounds according to the invention have PPAR-type receptor modulating properties. This activity on the PPARα, δ and γ receptors is measured in a transactivation test and quantified by the dissociation constant Kdapp (apparent), as described in Example 142.

The preferred compounds of the present invention have a dissociation constant of less than or equal to 1,000 nM, and advantageously of less than or equal to 500 nM.

The present invention also features medicaments containing the compounds of formula (I) as described above.

The present invention also features formulating the compounds of formula (I) into compositions suited for regulating and/or restoring the metabolism of skin lipids.

The compounds according to the invention are particularly suitable in the fields of the following treatments:
1) for treating dermatological conditions or afflictions linked to a keratinization disorder related to cell differentiation and proliferation, in particular to treat acne vulgaris, comedo-type acne, polymorphic acne, acne rosacea, nodulocystic acne, acne conglobata, senile acne, secondary acne such as solar acne, acne medicamentosa or occupational acne;
2) for treating other types of keratinization disorders, in particular ichthyosis, ichthyosiform states, Darrier's disease, keratosis palmaris et plantaris, leukoplasia and leukoplasiform states, cutaneous or mucosal (buccal) lichen;
3) for treating other dermatological conditions with an inflammatory immunoallergic component, with or without cell proliferation disorder, and in particular all the forms of psoriasis, whether cutaneous, mucosal or ungual, and even psoriatic rheumatism, or cutaneous atopy, such as eczema or respiratory atopy or gingival hypertrophy;
4) for treating any dermal or epidermal proliferations whether benign or malignant, whether of viral origin or not, such as verruca vulgaris, verruca plana and epidermodysplasia verruciformis, oral or florid papillomatoses, T lymphoma, and proliferations which may be induced by ultraviolet radiation, in particular in the case of baso- and spinocellular epitheliomas, and any precancerous skin lesions such as keratoacanthomas;
5) for treating other dermatological disorders such as immune dermatoses such as lupus erythematosus, bullous immune diseases and collagen diseases, such as scleroderma;
6) in the treatment of dermatological or general conditions with an immunological component;
7) in the treatment of skin disorders due to exposure to UV radiation and for repairing or combating skin aging, whether photoinduced or chronological or for reducing actinic keratoses and pigmentations, or any pathologies associated with chronological or actinic aging, such as xerosis;
8) for combating sebaceous function disorders such as acne hyperseborrhoea, simple seborrhea, or seborrhoeic dermatitis;
9) for preventing or treating cicatrization disorders, or for preventing or repairing stretch marks;
10) in the treatment of pigmentation disorders, such as hyperpigmentation, melasma, hypopigmentation or vitiligo;
11) in the treatment of lipid metabolism conditions, such as obesity, hyperlipidaemia, non-insulin-dependent diabetes or X syndrome;
12) in the treatment of inflammatory conditions such as arthritis;
13) in the treatment or prevention of cancerous or precancerous states;
14) in the prevention or treatment of alopecia of different origins, in particular alopecia due to chemotherapy or to radiation;
15) in the treatment of immune system disorders, such as asthma, diabetes mellitus type 1, multiple sclerosis, or other selective dysfunctions of the immune system; and
16) in the treatment of conditions of the cardiovascular system such as arteriosclerosis or hypertension.

The present invention also features pharmaceutical compositions comprising, formulated into a physiologically acceptable medium, at least one compound of formula (I) as defined above.

The administration (regime or regimen) of the compositions according to the invention may be carried out enterally, parenterally, topically or ocularly. Preferably, the pharmaceutical composition is packaged in a form suitable for application by the topical route.

By the enteral route, the composition may be provided in the form of tablets, gelatin capsules, sugar-coated tablets, syrups, suspensions, solutions, powders, granules, emulsions, suspensions of lipid or polymeric microspheres or nanospheres or vesicles allowing controlled release. By the parenteral route, the composition may be provided in the form of solutions or suspensions for perfusion or injection.

The compounds according to the invention are generally administered at a daily dose of about 0.001 mg/kg to 100 mg/kg of body weight, in 1 to 3 doses.

The compounds are administered by the systemic route at a concentration generally from 0.001% to 10% by weight, preferably from 0.01% to 1% by weight, relative to the weight of the composition.

By the topical route, the pharmaceutical compositions according to the invention are more particularly suited for the treatment of the skin and the mucous membranes and may be provided in the form of salves, creams, milks, ointments, powders, impregnated pads, syndets, solutions, gels, sprays, mousses, suspensions, lotions, sticks, shampoos or washing bases. They may also be provided in the form of suspensions of lipid or polymeric microspheres or nanospheres or vesicles or of polymeric patches and of hydrogels allowing controlled release. This composition for the topical route may be provided in anhydrous form, in aqueous form or in the form of an emulsion.

The compounds are administered by the topical route at a concentration which is generally from 0.001% to 10% by weight, preferably from 0.01% to 1% by weight, relative to the total weight of the composition.

The compounds of formula (I) according to the invention also find application in the cosmetics field, in particular in body and hair care, and more particularly for regulating and/or restoring skin lipid metabolism.

This invention therefore also features cosmetic application of a composition comprising, in a physiologically acceptable carrier, at least one of the compounds of formula (I) for body or hair care.

The cosmetic compositions according to the invention containing, in a cosmetically acceptable carrier, at least one compound of formula (I) or one of its optical or geometric isomers or one of its salts, may be provided in particular in the form of a cream, a milk, a lotion, a gel, suspensions of lipid or polymeric microspheres or nanospheres or vesicles, impregnated pads, solutions, sprays, mousses, sticks, soaps, shampoos or washing bases.

The concentration of compound of formula (I) in the cosmetic composition is preferably from 0.001% to 3% by weight, relative to the total weight of the composition.

The pharmaceutical and cosmetic compositions as described above may in addition contain inert additives, or even pharmacodynamically active additives as regards the pharmaceutical compositions, or combinations of these additives, and in particular:
wetting agents;
flavor enhancers;
preservatives such as esters of parahydroxybenzoic acid;
stabilizers;
moisture regulators;
pH regulators;
cosmetic pressure modifiers;
emulsifiers;
UV-A and UV-B screening agents;
antioxidants, such as α-tocopherol, butylated hydroxyanisole or butylated hydroxytoluene, Super Oxide Dismutase, Ubiquinol or certain metal chelators;
depigmenting agents such as hydroquinone, azelaic acid, caffeic acid or kojic acid;
emollients;
moisturizing agents such as glycerol, PEG 400, thiamorpholinone, and its derivatives, or urea;
antiseborrhoeic or anti-acne agents, such as S-carboxymethylcysteine, S-benzylcysteamine, their salts or their derivatives, or benzoyl peroxide;
antibiotics such as erythromycin and its esters, neomycin, clindamycin and its esters, tetracyclines;
antifungal agents such as ketoconazole or 4,5-polymethylene-3-isothiazolidones;
agents promoting hair regrowth, such as Minoxidil (2,4-diamino-6-piperidinopyrimidine 3-oxide) and its derivatives, Diazoxide (7-chloro-3-methyl-1,2,4-benzothiadiazine 1,1-dioxide) and Phenyloin (5,4-diphenylimidazolidine 2,4-dione);
nonsteroidal anti-inflammatory agents;
carotenoids and, in particular, β-carotene;
antipsoriatic agents such as anthralin and its derivatives; 5,8,11,14-eicosatetraynoic and 5,8,11-eicosatriynoic acids, their esters and amides;
retinoids, that is to say ligands for the RAR or RXR receptors, which may be natural or synthetic;
corticosteroids or oestrogens;
α-hydroxy acids and α-keto acids or their derivatives, such as lactic, malic, citric, glycolic, mandelic, tartaric, glyceric and ascorbic acids, and their salts, amides or esters, or β-hydroxy acids or their derivatives, such as salicylic acid and its salts, amides or esters;
ion channel, such as potassium channel, blockers;
or alternatively, more particularly for pharmaceutical compositions, in combination with medicaments known to interfere with the immune system (for example cyclosporine, FK 506, glucocorticoids, monoclonal antibodies, cytokines or growth factors, and the like).

Of course, one skilled in the art will be careful to choose the possible compound(s) to be added to these compositions such that the advantageous properties intrinsically associated with the present invention are not or not substantially impaired by the addition envisaged.

In order to further illustrate the present invention and the advantages thereof, the following specific examples are given, including those relating to the preparation of the compounds (I) as well as the biological activity and particular formulations thereof, it being understood that same are intended only as illustrative and in nowise limitative. In said examples to follow, all parts and percentages are given by weight, unless otherwise indicated.

### EXAMPLES

The products were analyzed by HPLC/Mass. Column: 2.1×5 mm, 3 μ, High purity C18 Hypersil.

Mobile phase: A (CH₃CN/0.1 v/v HCO₂H); B (H₂O/0.1 v/v HCO₂H), Waters Alliance 2790 LC Mobile Phase

| | | |
|---|---|---|
| Solvents | A % | 35.0 Solvent A |
| | B % | 65.0 Solvent B |

| | |
|---|---|
| Flow rate (ml/min) | 0.450 |
| Analytical time (min) | 5.00 |
| Column temperature (° C.) | 60 |
| Maximum column temperature (° C.) | 10 |
| Waters Alliance 2790 LC Rapid Equilibration | |
| System time (min) | 0.30 |
| Re-equilibration time (min) | 0.50 |

The gradient contains 3 entries which are:

| Time | A % | B % | Flow rate | Curve |
|---|---|---|---|---|
| 0.00 | 5.0 | 65.0 | 0.450 | 1 |
| 3.00 | 95.0 | 5.0 | 0.450 | 6 |
| 5.00 | 95.0 | 5.0 | 0.450 | 6 |

### Example 1

### Synthesis of ethyl 4-(3-aminophenylsulfanyl)phenyl]acetate

### a) Preparation of ethyl 4-iodophenylacetate

1.25 ml (0.023 mol) of concentrated sulfuric acid are added dropwise to a mixture of 6.14 g (0.023 mol) of 4-iodophenylacetic acid in 50 ml of ethanol. The reaction medium is then heated under reflux for 7 h, and then concentrated in a rotary evaporator under vacuum. Water is added to the residue obtained. The solution is neutralized by adding sodium bicarbonate. The desired product is extracted by adding ethyl ether. The organic phase is washed with water, dried over magnesium sulfate and concentrated in a rotary evaporator. The product is purified by filtration on a silica column, eluted with a dichloromethane 8/heptane 2 mixture. After evaporation of the solvents, 6.2 g (96%) of the expected compound are recovered in the form of a colorless oil.

### b) Synthesis of ethyl [4-(3-aminophenylsulfanyl)phenyl]acetate

A solution of 3-aminothiophenol (2 g, 0.016 mol) in 30 ml of THF is added over a mixture of borohydride polymer supported Amberlite® IRA400 resin (2.5 mmol/g) (Aldrich: 32864-2) (16.2 g, 0.04 mol), bis(bipyridine)nickel (II) bromide (150 mg) (Organometallics 1985, 4, 657-661) and ethyl 4-iodophenylacetate (3 g, 0.011 mol) in ethanol (120 ml). The mixture is stirred under reflux for 3 h and 12 h at room temperature. The reaction medium is filtered and the filtrate concentrated in a rotary evaporator under vacuum. The product is purified by chromatography on a silica column (dichloromethane 5/heptane 5). After evaporation of the solvents, the expected compound 2.2 g (70%), is isolated in the form of a yellow oil.

¹H NMR (CDCl₃, 400 MHz): 1.28 (3H, t), 3.61 (2H, s), 4.18 (2H, q), 6.57 (1H, Ar, d), 6.66 (1H, Ar, s), 6.75 (1H, Ar, d), 7.09 (1H, Ar, t), 7.23 (2H, Ar, d), 7.335 (2H, Ar, d).

### Example 2

### Synthesis of ethyl [4-(4-aminophenylsulfanyl)phenyl]acetate

In a manner similar to Example 1(b), by reacting ethyl 4-iodophenylacetate (2.5 g, 0.01 mol), 30 ml of THF, borohydride polymer supported Amberlitee IRA400 resin (2.5 mmol/g) (Aldrich: 32864-2) (13.5 g), bis(bipyridine)nickel (II) bromide (125 mg) (Organometallics 1985, 4, 657-661) and 4,4′-dithiodianiline (1.7 g, 0.013 mol), 1.1 g (42%) of the expected derivative is obtained in the form of a yellow oil.

¹H NMR (CDCl₃, 400 MHz): 1.26 (3H, t), 3.55 (2H, s), 4.15 (2H, q), 6.67 (2H, Ar, d), 7.10 (2H, Ar, d), 7.15 (2H, Ar, d), 7.32 (2H, Ar, d).

### Example 3

### Synthesis of {4-[3-(3-phenylpropylamino)phenyl-sulfanyl]phenyl}acetic acid

### a) Preparation of ethyl{4-[3-(3-phenylpropylamino)phenylsulfanyl]phenyl}acetate

A solution of 3-phenylpropionaldehyde (257 mg, 1.91 mmol) and acetic acid (1 ml) is added to a solution of ethyl [4-(3-aminophenylsulfanyl)phenyl]acetate (Example 1 (b)) (550 mg, 1.91 mmol) in 15 ml of DMF. DMF 241 mg and sodium cyanoborohydride (3.83 mmol) are added and the mixture is stirred for 12 h at room temperature. After extracting with ethyl ether, the organic phase is washed with water, dried over magnesium sulfate and concentrated in a rotary evaporator under vacuum. The product is purified by chromatography on a silica column (dichloromethane 7/heptane 3). After evaporation of the solvents, 601 mg (77%) of the expected derivative and 100 mg (10%) of ethyl(4-{3-[bis(3-phenylpropyl)amino]phenylsulfanyl}phenyl)acetate are obtained.

¹H NMR (CDCl₃, 400 MHz): 1.29 (3H, t), 1.91 to 1.99 (2H, m), 2.74 (2H, t), 3.13 (2H, t), 3.63 (2H, s), 3.69 (1H, NH, s), 4.20 (2H, q), 6.5 (1H, Ar, d), 6.61 (1H, Ar, s), 6.71 (1H, Ar, d), 7.11 (1H, Ar, t), 7.21 to 7.26 (5H, Ar, m), 7.33 (4H, Ar, t).

### b) Synthesis of {4-[3-(3-phenylpropylamino)phenylsulfanyl]phenyl}acetic acid

In a manner similar to Example 1(c), the subject compound is obtained by reacting ethyl{4-[3-(3-phenyl-propylamino)phenylsulfanyl]phenyl}acetate (Example 3(a)) (50 mg), sodium hydroxide (80 mg), water (500 μl) and ethanol (500 μl) in THF (3 ml).

### Example 4

### Synthesis of (4-{3-[bis(3-phenylpropyl)amino]phenylsulfanyl}phenyl)acetic acid

In a manner similar to Example 1(c), the subject compound is obtained by reacting ethyl (4-{3-[bis(3-phenylpropyl)amino]phenylsulfanyl}phenyl)acetate obtained in (Example 3(a)) (50 mg), sodium hydroxide (80 mg), water (500 μl) and ethanol (500 μl) in THF (3 ml).

### Example 5

### Synthesis of [4-(3-phenylethylamino)phenylsulfanylphenyl]acetic acid

### a) Preparation of ethyl [4-(3-phenylethylamino)phenylsulfanylphenyl]acetate

In a manner similar to Example 3(a), by reacting 3-phenylacetaldehyde (230 mg, 1.91 mmol), acetic acid (1 ml), ethyl [4-(3-aminophenylsulfanyl)phenyl]acetate (Example 1 (b)) (550 mg, 1.91 mmol) in 15 ml of DMF and 241 mg of sodium cyanoborohydride (3.83 mmol), 643 mg (86%) of the expected derivative are obtained in the form of a colorless oil and 26 mg (10%) of ethyl [4-(3-diphenethylaminophenylsulfanyl)phenyl]acetate.

### b) Synthesis of [4-(3-phenethylamino)phenylsulfanylphenyl]acetic acid

In a manner similar to Example 1(c), the subject compound is obtained by reacting ethyl [4-(3-phenethyl-amino)phenylsulfanylphenyl]acetate (Example 5(a)) (50 mg), sodium hydroxide (80 mg), water (500 μl) and ethanol (500 μl) in THF (3 ml).

### Example 6

### Synthesis of [4-(3-diphenethylaminophenylsulfanyl)phenyl]acetic acid

In a manner similar to Example 1(c), the subject compound is obtained by reacting ethyl [4-(3-diphenethylaminophenylsulfanyl)phenyl]acetate obtained in Example 5(a) (50 mg), sodium hydroxide (80 mg), water (500 μl) and ethanol (500 μl) in THF (3 ml).

### Example 7

### Synthesis of [4-(3-heptylaminophenylsulfanyl)phenyl]acetic acid

### a) Preparation of ethyl [4-(3-heptylaminophenylsulfanyl)phenyl]acetate

In a manner similar to Example 3(a), by reacting heptaldehyde (219 mg, 1.91 mmol), acetic acid (1 ml), ethyl [4-(3-aminophenylsulfanyl)phenyl]acetate (Example 1(b)) (550 mg, 1.91 mmol) in 15 ml of DMF and 241 mg of sodium cyanoborohydride (2.71 mmol), 330 mg (45%) of the expected derivative are obtained in the form of a colorless oil and 64 mg (7%) of ethyl [4-(3-diheptylaminophenylsulfanyl)phenyl]acetate.

¹H NMR (CDCl₃, 400 MHz): 0.93 (3H, t), 1.28 (3H, t), 1.32 to 1.41 (8H, m), 1.57 to 1.64 (2H, m), 3.07 (2H, t), 3.06 (2H, t), 3.60 (2H, s), 4.18 (2H, q), 6.49 (1H, Ar, d), 6.61 (1H, Ar, s), 6.68 (1H, Ar, d), 7.11 (1H, Ar, t), 7.23 (2H, Ar, d), 7.32 (2H, Ar, d).

### b) Synthesis of [4-(3-heptylaminophenylsulfanyl)phenyl]acetic acid

In a manner similar to Example 1(c), the subject compound is obtained by reacting ethyl [4-(3-heptylaminophenylsulfanyl)phenyl]acetate (Example 7(a)) (50 mg), sodium hydroxide (80 mg), water (500 μl) and ethanol (500 μl) in THF (3 ml).

### Example 8

### Synthesis of [4-(3-diheptylaminophenylsulfanyl)phenyl]acetic acid

In a manner similar to Example 1(c), the subject compound is obtained by reacting ethyl [4-(3-diheptylaminophenylsulfanyl)phenyl]acetate obtained in Example 7(a) (50 mg), sodium hydroxide (80 mg), water (500 LI) and ethanol (500 μl) in THF (3 ml).

### Example 9

### Synthesis of [4-(3-butylaminophenylsulfanyl)phenyl]acetic acid

### a) Preparation of ethyl [4-(3-butylaminophenylsulfanyl)phenyl]acetate

In a manner similar to Example 3(a), by reacting butyraldehyde (138 mg, 1.91 mmol), acetic acid (1 ml), ethyl [4-(3-aminophenylsulfanyl)phenyl]acetate (Example 1(b)) (550 mg, 1.91 mmol) in 15 ml of DMF and 241 mg of sodium cyanoborohydride (2.71 mmol), 140 mg (21%) of the expected derivative are obtained in the form of a colorless oil and 464 mg (61%) of ethyl [4-(3-dibutylaminophenylsulfanyl)phenyl]acetate.

¹H NMR (CDCl₃, 400 MHz): 0.97 (3H, t), 1.28 (3H, t), 1.39 to 1.45 (2H, m), 1.57 to 1.61 (2H, m), 3.08 (2H, t), 3.62 (2H, s), 3.67 (1H, NH, s), 4.12 (2H, q), 6.51 (1H, Ar, d), 6.61 (1H, Ar, s), 6.69 (1H, Ar, d), 7.11 (1H, Ar, t), 7.23 (2H, Ar, d), 7.33 (2H, Ar, d).

### b) Synthesis of [4-(3-butylaminophenylsulfanyl)phenyl]acetic acid

In a manner similar to Example 1(c), the subject compound is obtained by reacting ethyl [4-(3-butylaminophenylsulfanyl)phenyl]acetate (Example 9(a)) (50 mg), sodium hydroxide (80 mg), water (500 μl) and ethanol (500 μl) in THF (3 ml).

### Example 10

### Synthesis of [4-(3-dibutylaminophenylsulfanyl)phenyl]acetic acid

In a manner similar to Example 1(c), the subject compound is obtained by reacting ethyl [4-(3-dibutylaminophenylsulfanyl)phenyl]acetate obtained in Example 9(a) (50 mg, 0.174 mmol), sodium hydroxide (80 mg), water (500 μl) and ethanol (500 μl) in THF (3 ml).

### Example 11

### Synthesis of {4-[4-(3-phenylpropylamino)phenylsulfanyl]phenyl}acetic acid

### a) Preparation of ethyl {4-[4-(3-phenylpropylamino)phenylsulfanyl]phenyl}acetate

In a manner similar to Example 3(a), by reacting 3-phenylpropionaldehyde (128 mg, 0.96 mmol), acetic acid (1 ml), ethyl [4-(4-aminophenylsulfanyl)phenyl]acetate (Example 2(a)) (275 mg, 0.96 mmol) in 15 ml of DMF 120 mg and sodium cyanoborohydride (1.91 mmol), 307 mg (79%) of the expected derivative and 55 mg (11%) of ethyl(4-{4-[bis(3-phenylpropyl)amino]phenylsulfanyl}phenyl)acetate are obtained.

¹H NMR (CDCl₃, 400 MHz): 1.27 (3H, t), 1.96 to 2.03 (2H, m), 2.77 (2H, t), 3.19 (2H, t), 3.56 (2H, s), 3.86 (1H, NH, s), 4.16 (2H, q), 6.58 (1H, Ar, d), 7.09 (2H, Ar, d), 7.15 (2H, Ar, d), 7.23 to 7.26 (3H, Ar, m), 7.32 to 7.36 (4H, Ar, m).

### b) Synthesis of {4-[4-(3-phenylpropylamino)phenylsulfanyl]phenyl}acetic acid

In a manner similar to Example 1(c), the subject compound is obtained by reacting ethyl{4-[4-(3-phenyl-propylamino)phenylsulfanyl]phenyl}acetate (Example 11(a)) (50 mg), sodium hydroxide (80 mg), water (500 μl) and ethanol (500 μl) in THF (3 ml).

### Example 12

### Synthesis of (4-{4-[bis(3-phenylpropyl)amino]phenylsulfanyl}phenyl)acetic acid

In a manner similar to Example 1(c), the subject compound is obtained by reacting ethyl (4-{4-[bis(3-phenylpropyl)amino]phenylsulfanyl}phenyl)acetate obtained in Example 11 (a) (50 mg), sodium hydroxide (80 mg), water (500 μl) and ethanol (500 μl) in THF (3 ml).

### Example 13

### Synthesis of [4-(4-phenethylaminophenylsulfanyl)phenyl]acetic acid

### a) Preparation of ethyl [4-(4-phenethylaminophenylsulfanyl)phenyl]acetate

In a manner similar to Example 3(a), by reacting phenylacetaldehyde (115 mg, 0.96 mmol), acetic acid (1 ml), ethyl [4-(4-aminophenylsulfanyl)phenyl]acetate (Example 2(a)) (275 mg, 0.96 mmol) in 15 ml of DMF and 241 mg of sodium cyanoborohydride (3.83 mmol), 311 mg (83%) of the expected derivative are obtained in the form of a colorless oil and 17 mg (4%) of ethyl [4-(4-diphenethylaminophenylsulfanyl)phenyl]acetate.

¹H NMR (CDCl₃, 400 MHz): 1.29 (3H, t), 2.97 (2H, t), 3.45 (2H, t), 3.58 (2H, s), 3.95 (1H, NH, s), 4.18 (2H, q), 6.32 (2H, Ar, d), 7.12 (2H, Ar, d), 7.18 (2H, Ar, d), 7.27 to 7.30 (3H, Ar, m), 7.36 to 7.40 (4H, Ar, m).

### b) Synthesis of [4-(4-phenethylaminophenylsulfanyl)phenyl]acetic acid

In a manner similar to Example 1(c), the subject compound is obtained by reacting ethyl [4-(4-phenethyl-aminophenylsulfanyl)phenyl]acetate (Example 13(a)) (50 mg), sodium hydroxide (80 mg), water (500 μl) and ethanol (500 μl) in THF (3 ml).

### Example 14

### Synthesis of [4-(4-diphenethylaminophenylsulfanyl)phenyl]acetic acid

In a manner similar to Example 1(c), the subject compound is obtained by reacting ethyl [4-(4-diphenethylaminophenylsulfanyl)phenyl]acetate obtained in Example 13(a) (50 mg), sodium hydroxide (80 mg), water (500 μl) and ethanol (500 μl) in THF (3 ml).

### Example 15

### Synthesis of [4-(4-heptylaminophenylsulfanyl)phenyl]acetic acid

### a) Preparation of ethyl [4-(4-heptylaminophenylsulfanyl)phenyl]acetate

In a manner similar to Example 3(a), by reacting heptaldehyde (109 mg, 0.96 mmol), acetic acid (1 ml), ethyl [4-(4-aminophenylsulfanyl)phenyl]acetate (Example 2(a)) (275 mg, 0.96 mmol) in 15 ml of DMF and 241 mg of sodium cyanoborohydride (2.71 mmol), 250 mg (68%) of the expected derivative are obtained in the form of a colorless oil and 43 mg (9%) of ethyl [4-(4-diheptylaminophenylsulfanyl)phenyl]acetate.

¹H NMR (CDCl₃, 400 MHz): 0.92 (3H, t), 1.28 (3H, t), 1.30 to 1.43 (8H, m), 1.63 to 1.67 (2H, m), 3.14 (2H, t), 3.55 (2H, s), 3.86 (1H, NH, s), 4.15 (2H, q), 6.60 (2H, Ar, d), 7.08 (2H, Ar, d), 7.15 (2H, Ar, d), 7.35 (2H, Ar, d).

### b) Synthesis of [4-(4-heptylaminophenylsulfanyl)phenyl]acetic acid

In a manner similar to Example 1(c), the subject compound is obtained by reacting ethyl [4-(4-heptylaminophenylsulfanyl)phenyl]acetate (Example 15(a)) (50 mg), sodium hydroxide (80 mg), water (500 μl) and ethanol (500 μl) in THF (3 ml).

### Example 16

### Synthesis of [4-(4-diheptylaminophenylsulfanyl)phenyl]acetic acid

In a manner similar to Example 1(c), the subject compound is obtained by reacting ethyl [4-(4-diheptylaminophenylsulfanyl)phenyl]acetate obtained in Example 15(a) (50 mg), sodium hydroxide (80 mg), water (500 μl) and ethanol (500 μl) in THF (3 ml).

### Example 17

### Synthesis of [4-(4-dibutylaminophenylsulfanyl)phenyl]acetic acid

### a) Preparation of ethyl [4-(4-dibutylaminophenylsulfanyl)phenyl]acetate

In a manner similar to Example 3(a), by reacting butyraldehyde (69 mg, 0.96 mmol), acetic acid (1 ml), ethyl [4-(4-aminophenylsulfanyl)phenyl]acetate (Example 2(a)) (275 mg, 0.96 mmol) in 15 ml of DMF and 241 mg of sodium cyanoborohydride (2.71 mmol), 313 mg (82%) of ethyl [4-(4-dibutylaminophenylsulfanyl)-phenyl]acetate are obtained.

### b) Synthesis of [4-(4-dibutylaminophenylsulfanyl)phenyl]acetic acid

In a manner similar to Example 1(c), the subject compound is obtained by reacting ethyl [4-(4-dibutylaminophenylsulfanyl)phenyl]acetate (Example 17(a)) (50 mg), sodium hydroxide (80 mg), water (500 μl) and ethanol (500 μl) in THF (3 ml).

TABLE 1
Results of analysis of the compounds of Examples 1b
to 17b:

| | | | HPLC | ES Mass |
|---|---|---|---|---|
| | Quantity | Molar | (% total | Spectrometry |
| EXAMPLE | (mg) | mass | surface area) | (M + H⁺) |
| 1b | 1.4 | 259.33 | 100 | 259 |
| 2b | 1.7 | 259.33 | 72.96 | 259 |
| 3b | 178.6 | 377.51 | 89.68 | 378 |
| 4b | 49.3 | 495.68 | 82.13 | 496 |
| 5b | 157.6 | 363.48 | 75.62 | 363 |
| 6b | 13.4 | 467.63 | 73.84 | 468 |
| 7b | 20.4 | 357.52 | 93.81 | 358 |
| 8b | 29.9 | 455.70 | 98.15 | 456 |
| 9b | 4.1 | 315.43 | 72.45 | 315 |
| 10b | 93.3 | 371.54 | 94.21 | 372 |
| 11b | 19.2 | 377.51 | 95.56 | 378 |
| 12b | 34.1 | 495.68 | 82.98 | 496 |
| 13b | 21.4 | 363.48 | 89.16 | 363 |
| 14b | 6.7 | 467.63 | 87.55 | 468 |
| 15b | 3.6 | 357.52 | 89.2 | 358 |
| 16b | 26.1 | 455.70 | 95.67 | 456 |
| 17b | 125.4 | 371.54 | 92.31 | 372 |

### EXAMPLES 18 TO 141: SYNTHESIS OF EXAMPLES 18 to 141:

Examples 18 to 141 were obtained by parallel chemistry. The reactions of a starting amine and a starting isocyanate are performed in several reactors simultaneously according to the operating protocol described below.

### A. Formation of urea for the [4-(aminophenylsulfanyl)-phenyl]acetic acid derivatives

Operating Protocol:

The starting amine (see Table 3) is introduced into each 5 ml reactor. 2 ml of dichloromethane are added. Next, 0.062 mmol of isocyanate (see Table 4) are added. The reactors are stirred for 7 h at room temperature. 0.062 mmol of isocyanates are added if the starting amine has not completely disappeared (TLC monitoring). In this case, the stirring is continued for 12 h at room temperature.

The reaction media are concentrated to dryness for 2 h at 40° C. in a centrifugal evaporator under vacuum. The products are purified by filtration on silica cartridges (6 ml), 1:DCM, 2:DCM 80/AcOEt 20, and then concentrated to dryness, 2 h at 40° C. in a centrifugal evaporator.

TABLE 2
Starting amines:

| Ex- | | | Number | Quantity |
|---|---|---|---|---|
| ample | | Molar | of mol | per |
| No. | Name | mass | (mmol) | reactor |
| 3a | Ethyl {4-[3-(3-phenyl- | 405.56 | 0.031123 | 12.6 |
| | propylamino)phenylsulfanyl] | | | |
| | phenyl}acetate | | | |
| 5a | Ethyl [4-(3-phenethyl- | 391.53 | 0.031123 | 12.2 |
| | amino)phenylsulfanyl- | | | |
| | phenyl]acetate | | | |
| 7a | Ethyl [4-(3-heptyl- | 385.57 | 0.031123 | 12.0 |
| | aminophenylsulfanyl)- | | | |
| | phenyl]acetate | | | |
| 9a | Ethyl [4-(3-butyl- | 343.49 | 0.018923 | 6.5 |
| | aminophenylsulfanyl)- | | | |
| | phenyl]acetate | | | |
| 11a | Ethyl {4-[4-(3-phenyl- | 405.56 | 0.031123 | 12.6 |
| | propylamino)phenylsulfanyl] | | | |
| | phenyl}acetate | | | |
| 13a | Ethyl [4-(4-phenethyl- | 391.53 | 0.031123 | 12.2 |
| | aminophenylsulfanyl)- | | | |
| | phenyl]acetate | | | |
| 15a | Ethyl [4-(4-heptyl- | 385.57 | 0.031123 | 12.0 |
| | aminophenylsulfanyl)- | | | |
| | phenyl]acetate | | | |

TABLE 3
Starting isocyanates:

| | | | n (mmol) | Mol | m | V |
|---|---|---|---|---|---|---|
| CHEMISTRY | Structure | MW | 2 eq | equivalent | (mg) | (miroL) |
| BENZYL ISOCYANATE | 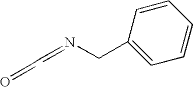 | 133.15 | 0.0622 | 2 | 8.3 | 8 |
| PHENYL ISOCYANATE | 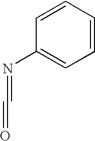 | 119.12 | 0.0622 | 2 | 7.4 | 7 |
| 2,3-DICHLOROPHENYL ISOCYANATE | 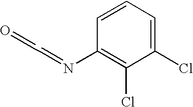 | 188.01 | 0.0622 | 2 | 11.7 | |
| HEPTYL ISOCYANATE | 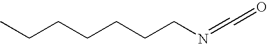 | 141.21 | 0.0622 | 2 | 8.8 | 10 |
| PHENETHYL ISOCYANATE | 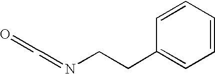 | 147.18 | 0.0622 | 2 | 9.2 | 9 |
| 4-(TRIFLUOROMETHYL)PHENYL ISOCYANATE | 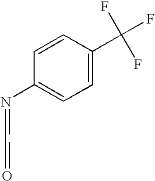 | 187.12 | 0.0622 | 2 | 11.6 | |
| 4-METHOXYPHENYL ISOCYANATE | 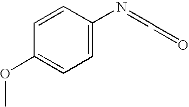 | 149.15 | 0.0622 | 2 | 9.3 | |
| 1-ADAMANTYL ISOCYANATE | 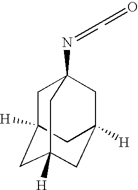 | 177.25 | 0.0622 | 2 | 11.0 | |
| 2-PHENOXYPHENYL ISOCYANATE | 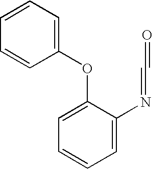 | 211.22 | 0.0622 | 2 | 13.1 | 11 |
| ALLYL ISOCYANATE | 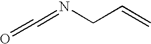 | 83.09 | 0.0622 | 2 | 5.2 | 6 |
| CYCLOHEXYL ISOCYANATE | 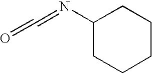 | 125.17 | 0.0622 | 2 | 7.8 | 8 |
| 2-NITROPHENYL ISOCYANATE | 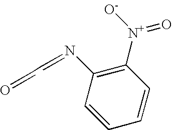 | 164.12 | 0.0622 | 2 | 10.2 | |
| HEXYL ISOCYANATE | 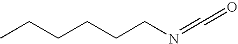 | 127.19 | 0.0622 | 2 | 7.9 | 9 |
| 2-NAPHTHYL ISOCYANATE | 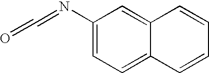 | 169.18 | 0.0622 | 2 | 10.5 | |
| 2-ETHOXYPHENYL ISOCYANATE | 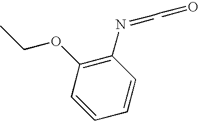 | 163.18 | 0.0622 | 2 | 10.2 | 9 |
| 4-BUTOXYPHENYL ISOCYANATE | 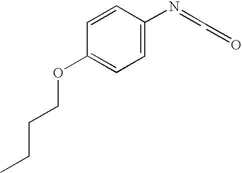 | 191.23 | 0.0622 | 2 | 11.9 | 11 |
| PENTYL ISOCYANATE | 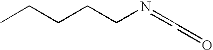 | 113.16 | 0.0622 | 2 | 7.0 | 8 |
| N-BUTYL ISOCYANATE | 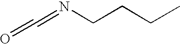 | 99.13 | 0.0622 | 2 | 6.2 | 7 |
| 4-(DIMETHYLAMINO)PHENYL ISOCYANATE | 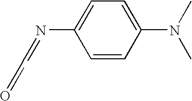 | 162.19 | 0.0622 | 2 | 10.1 | |

B. Saponification:

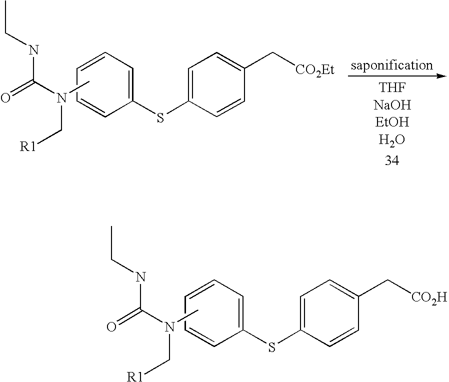

Each of the esters obtained above is solubilized in 2 ml of THF. 100 μl of ethanol are then introduced. 100 μl of a sodium hydroxide solution at 35% is then added. The mixture is stirred at room temperature for 48 h. The progress of the reaction is monitored by thin-layer chromatography (DCM 80/AcOEt 20). After extracting with ether, acidifying with a 1N hydrochloric acid solution, the organic phase is washed twice with water, dried over magnesium sulfate and concentrated to dryness in a centrifugal evaporator under vacuum. The products are purified by filtration on silica cartridges (6 ml) if necessary, and then concentrated to dryness for 2 h at 40° C. in a centrifugal evaporator under vacuum. The final products are analyzed by mass-coupled HPLC.

TABLE 4
Analysis of the compounds of Examples 18b to 141b:

| | | | | | | HPLC | |
|---|---|---|---|---|---|---|---|
| | | | | | | (% | |
| Ex- | | | | Quan- | | total | ES |
| am- | | | | tity | | surface | MASS |
| ple | Amine | Isocyanate | Final product | (mg) | MW | area) | (M + H⁺) |
| 18b | Ethyl {4-[3-(3-phenylpropylamino) | benzyl | (4-{3-[3-Benzyl-1-(3- | 15.6 | 510.66 | 81.3 | 511 |
| | phenylsulfanyl] | isocyanate | phenylpropyl)- | | | | |
| | phenyl}acetate | | ureido]phenylsulfanyl}- | | | | |
| | | | phenyl]acetic acid | | | | |
| 19b | Ethyl {4-[3-(3-phenylpropylamino) | phenyl | (4-{3-[3-Phenyl-1-(3- | 22.6 | 496.63 | 89.21 | 497 |
| | phenylsulfanyl] | isocyanate | phenylpropyl)ureido]- | | | | |
| | phenyl}acetate | | phenylsulfanyl}phenyl)acetic | | | | |
| | | | acid | | | | |
| 20b | Ethyl {4-[3-(3-phenylpropylamino) | 2,3- | (4-{3-[3-(2,3-Dichlorophenyl)- | 15.7 | 565.52 | 60.51 | 566 |
| | phenylsulfanyl] | dichlorophenyl | 1-(3-phenylpropyl)- | | | | |
| | phenyl}acetate | isocyanate | ureido]phenylsulfanyl}phenyl) | | | | |
| | | | acetic acid | | | | |
| 21b | Ethyl {4-[3-(3-phenylpropylamino) | heptyl | (4-{3-[3-Heptyl-1-(3- | 19.4 | 518.72 | 85.44 | 519 |
| | phenylsulfanyl] | isocyanate | phenylpropyl)ureido]- | | | | |
| | phenyl}acetate | | phenylsulfanyl}phenyl)acetic | | | | |
| | | | acid | | | | |
| 22b | Ethyl {4-[3-(3-phenylpropylamino) | phenethyl | (4-{3-[3-Phenethyl-1-(3- | 15.1 | 524.68 | 79.28 | 525 |
| | phenylsulfanyl] | isocyanate | phenylpropyl)ureido]- | | | | |
| | phenyl}acetate | | phenylsulfanyl}phenyl)acetic | | | | |
| | | | acid | | | | |
| 23b | Ethyl {4-[3-(3-phenylpropylamino) | 4-(trifluoromethyl) | (4-{3-[1-(3-Phenylpropyl)-3-(4- | 4.9 | 564.63 | 75.5 | 565 |
| | phenylsulfanyl] | phenyl | trifluoromethylphenyl) | | | | |
| | phenyl}acetate | isocyanate | ureido]phenylsulfanyl} | | | | |
| | | | phenyl)acetic acid | | | | |
| 24b | Ethyl {4-[3-(3-phenylpropylamino) | 4-methoxyphenyl | (4-{3-[3-(4-Methoxyphenyl)-1- | 19.0 | 526.65 | 93.51 | 527 |
| | phenylsulfanyl] | isocyanate | (3-phenylpropyl)- | | | | |
| | phenyl}acetate | | ureido]phenylsulfanyl}phenyl)- | | | | |
| | | | acetic acid | | | | |
| 25b | Ethyl {4-[3-(3-phenylpropylamino) | 1-adamantyl | (4-{3-[Adamantan-1-yl-1-(3- | 11.8 | 554.75 | 35.5 | 555 |
| | phenylsulfanyl] | isocyanate | phenylpropyl)ureido]- | | | | |
| | phenyl}acetate | | phenylsulfanyl}phenyl)acetic | | | | |
| | | | acid | | | | |
| 26b | Ethyl {4-[3-(3-phenylpropylamino) | 2-phenoxyphenyl | (4-{3-[3-(2-Phenoxyphenyl)-1- | 19.4 | 588.73 | 87.33 | 589 |
| | phenylsulfanyl] | isocyanate | (3-phenylpropyl)- | | | | |
| | phenyl}acetate | | ureido]phenylsulfanyl}phenyl)- | | | | |
| | | | acetic acid | | | | |
| 27b | Ethyl {4-[3-(3-phenylpropylamino) | allyl | (4-{3-[3-Allyl-1-(3- | 15.7 | 460.60 | 89.29 | 461 |
| | phenylsulfanyl] | isocyanate | phenoxypropyl)ureido]phenylsulfanyl} | | | | |
| | phenyl}acetate | | phenyl)acetic acid | | | | |
| 28b | Ethyl {4-[3-(3-phenylpropylamino) | cyclohexyl | (4-{3-[3-Cyclohexyl-1-(3- | 14.8 | 502.68 | 57.7 | 503 |
| | phenylsulfanyl] | isocyanate | phenylpropyl)ureido]- | | | | |
| | phenyl}acetate | | phenylsulfanyl}phenyl)acetic | | | | |
| | | | acid | | | | |
| 29b | Ethyl {4-[3-(3-phenylpropylamino) | 2-nitrophenyl | (4-{3-[3-(2-Nitrophenyl)-1-(3- | 17.7 | 541.63 | 92 | 542 |
| | phenylsulfanyl] | isocyanate | phenylpropyl)ureido]- | | | | |
| | phenyl}acetate | | phenylsulfanyl}phenyl)acetic | | | | |
| | | | acid | | | | |
| 30b | Ethyl {4-[3-(3-phenylpropylamino) | hexyl | (4-{3-[3-Hexyl-1-(3- | 21.1 | 504.69 | 86.67 | 505 |
| | phenylsulfanyl] | isocyanate | phenylpropyl)ureido]phenylsulfanyl} | | | | |
| | phenyl}acetate | | phenyl)acetic acid | | | | |
| 31b | Ethyl {4-[3-(3-phenylpropylamino) | 2-naphthyl | (4-{3-[3-Naphthalen-2-yl-1-(3- | 19.5 | 546.69 | 79.59 | 547 |
| | phenylsulfanyl] | isocyanate | phenylpropyl)ureido]- | | | | |
| | phenyl}acetate | | phenylsulfanyl}phenyl)acetic | | | | |
| | | | acid | | | | |
| 32b | Ethyl {4-[3-(3-phenylpropylamino) | 2- | (4-{3-[3-(2-Ethoxyphenyl)-1-(3- | 20.0 | 540.68 | 87.68 | 541 |
| | phenylsulfanyl] | ethoxyphenyl | phenylpropyl)ureido]- | | | | |
| | phenyl}acetate | isocyanate | phenylsulfanyl}phenyl)acetic | | | | |
| | | | acid | | | | |
| 33b | Ethyl {4-[3-(3-phenylpropylamino) | 4- | (4-{3-[3-(4-Butoxyphenyl)-1-(3- | 5.8 | 568.74 | 80.69 | 569(prep) |
| | phenylsulfanyl] | butoxyphenyl | phenylpropyl)- | | | | |
| | phenyl}acetate | isocyanate | ureido]phenylsulfanyl}phenyl) | | | | |
| | | | acetic acid | | | | |
| 34b | Ethyl {4-[3-(3-phenylpropylamino) | pentyl | (4-{3-[3-Pentyl-1-(3- | 17.1 | 490.67 | 96.36 | 491 |
| | phenylsulfanyl] | isocyanate | phenylpropyl)ureido]phenylsulfanyl} | | | | |
| | phenyl}acetate | | phenyl)acetic acid | | | | |
| 35b | Ethyl {4-[3-(3-phenylpropylamino) | n-butyl | (4-{3-[3-Butyl-1-(3- | 33.4 | 476.64 | 82.58 | 477 |
| | phenylsulfanyl] | isocyanate | phenylpropyl)ureido]phenylsulfanyl} | | | | |
| | phenyl}acetate | | phenyl)acetic acid | | | | |
| 36b | Ethyl {4-[3-(3-phenylpropylamino) | 4-(dimethylamino) | (4-{3-[3-(4- | 16.8 | 539.70 | 60.24 | 540 |
| | phenylsulfanyl] | phenyl | Dimethylaminophenyl)-1-(3- | | | | |
| | phenyl}acetate | isocyanate | phenylpropyl)- | | | | |
| | | | ureido]phenylsulfanyl}phenyl) | | | | |
| | | | acetic acid | | | | |
| 37b | Ethyl [4-(3- | benzyl | {4-[3-(3-Benzyl-1- | 24.4 | 496.63 | 97.31 | 497 |
| | phenethylamino)phenylsulfanylphenyl] | isocyanate | phenethylureido)phenylsulfanyl] | | | | |
| | acetate | | phenyl}acetic acid | | | | |
| 38b | Ethyl [4-(3- | phenyl | {4-[3-(1-Phenethyl-3- | 14.0 | 482.60 | 74.12 | 483 |
| | phenethylamino)phenyl | isocyanate | phenylureido)phenylsulfanyl]- | | | | |
| | sulfanylphenyl]acetate | | phenyl}acetic acid | | | | |
| 39b | Ethyl [4-(3- | 2,3- | (4-{3-[3-(2,3-Dichlorophenyl)- | 16.6 | 551.49 | 79 | 551 |
| | phenethylamino)phenyl | dichlorophenyl | 1-phenethylureido]- | | | | |
| | sulfanylphenyl]acetate | isocyanate | phenylsulfanyl}phenyl)acetic | | | | |
| | | | acid | | | | |
| 40b | Ethyl [4-(3- | heptyl | {4-[3-(3-Heptyl-1- | 16.9 | 504.69 | 58.09 | 505 |
| | phenethylamino)phenyl | isocyanate | phenethylureido)phenylsulfanyl] | | | | |
| | sulfanylphenyl]acetate | | phenyl}acetic acid | | | | |
| 41b | Ethyl [4-(3- | phenethyl | {4-[3-(1,3- | 16.9 | 510.66 | 79.84 | 511 |
| | phenethylamino)phenyl | isocyanate | Diphenethylureido)phenylsulfanyl] | | | | |
| | sulfanylphenyl]acetate | | phenyl}acetic acid | | | | |
| 42b | Ethyl [4-(3- | 4-(trifluoro- | (4-{3-[1-phenethyl-3-(4- | 16.0 | 550.60 | 45.29 | 551 |
| | phenethylamino)phenyl | methyl)phenyl | trifluoromethylphenyl)- | | | | |
| | sulfanylphenyl]acetate | isocyanate | ureido]phenylsulfanyl}phenyl)- | | | | |
| | | | acetic acid | | | | |
| 43b | Ethyl [4-(3- | 4-methoxy- | (4-{3-[3-(4-Methoxyphenyl)-1- | 19.0 | 512.63 | 82.71 | 513 |
| | phenethylamino)phenyl | phenyl | phenethylureido]- | | | | |
| | sulfanylphenyl]acetate | isocyanate | phenylsulfanyl}phenyl)acetic | | | | |
| | | | acid | | | | |
| 44b | Ethyl [4-(3- | 1-adamantyl | {4-[3-(3-Adamantan-1-yl-1- | 14.9 | 540.73 | 52.66 | 541 |
| | phenethylamino)phenyl | isocyanate | phenethylureido)phenylsulfanyl] | | | | |
| | sulfanylphenyl]acetate | | phenyl}acetic acid | | | | |
| 45b | Ethyl [4-(3- | 2-phenoxy- | (4-{3-[1-Phenethyl-3-(2- | 2.9 | 574.70 | 95.43 | 575(prep) |
| | phenethylamino)phenyl | phenyl | phenoxyphenyl)ureido]- | | | | |
| | sulfanylphenyl]acetate | isocyanate | phenylsulfanyl}phenyl)acetic | | | | |
| | | | acid | | | | |
| 46b | Ethyl [4-(3- | allyl | {4-[3-(3-Allyl-1- | 16.0 | 446.57 | 59.31 | 447 |
| | phenethylamino)phenyl | isocyanate | phenethylureido)phenylsulfanyl] | | | | |
| | sulfanylphenyl]acetate | | phenyl}acetic acid | | | | |
| 47b | Ethyl [4-(3- | cyclohexyl | {4-[3-(3-Cyclohexyl-1- | 17.6 | 488.65 | 53.61 | 489 |
| | phenethylamino)phenyl | isocyanate | phenethylureido)phenylsulfanyl] | | | | |
| | sulfanylphenyl]acetate | | phenyl}acetic acid | | | | |
| 48b | Ethyl [4-(3- | 2-nitrophenyl | (4-{3-[3-(2-Nitrophenyl)-1- | 17.2 | 527.60 | 84 | 528 |
| | phenethylamino)phenyl | isocyanate | phenethylureido]phenylsulfanyl} | | | | |
| | sulfanylphenyl]acetate | | phenyl)acetic acid | | | | |
| 49b | Ethyl [4-(3- | hexyl | {4-[3-(3-Hexyl-1- | 16.3 | 490.67 | 76.67 | 491 |
| | phenethylamino)phenyl | isocyanate | phenethylureido)phenylsulfanyl] | | | | |
| | sulfanylphenyl]acetate | | phenyl}acetic acid | | | | |
| 50b | Ethyl [4-(3- | 2-naphthyl | {4-[3-(3-Naphthalen-2-yl-1- | 19.9 | 532.66 | 72.65 | 533 |
| | phenethylamino)phenyl | isocyanate | phenethylureido)phenylsulfanyl] | | | | |
| | sulfanylphenyl]acetate | | phenyl}acetic acid | | | | |
| 51b | Ethyl [4-(3- | 2- | (4-{3-[3-(2-Ethoxyphenyl)-1- | 16.3 | 526.65 | 78.93 | 527 |
| | phenethylamino)phenyl | ethoxyphenyl | phenethylureido]- | | | | |
| | sulfanylphenyl]acetate | isocyanate | phenylsulfanyl}phenyl)acetic | | | | |
| | | | acid | | | | |
| 52b | Ethyl [4-(3- | 4- | (4-{3-[3-(4-Butoxyphenyl)-1- | 19.2 | 554.71 | 60.45 | 555 |
| | phenethylamino)phenyl | butoxyphenyl | phenethylureido]- | | | | |
| | sulfanylphenyl]acetate | isocyanate | phenylsulfanyl}phenyl acetic | | | | |
| | | | acid | | | | |
| 53b | Ethyl [4-(3- | pentyl | {4-[3-(3-Pentyl-1- | 6.3 | 476.64 | 84.42 | 477(prep) |
| | phenethylamino)phenyl | isocyanate | phenethylureido)phenylsulfanyl] | | | | |
| | sulfanylphenyl]acetate | | phenyl}acetic acid | | | | |
| 54b | Ethyl [4-(3- | n-butyl | {4-[3-(3-Butyl-1- | 13.2 | 462.62 | 60.75 | 463 |
| | phenethylamino)phenyl | isocyanate | phethylureido)phenylsulfanyl]- | | | | |
| | sulfanylphenyl]acetate | | phenyl}acetic acid | | | | |
| 55b | Ethyl [4-(3- | 4-(dimethyl | (4-{3-[3-(4- | 9.3 | 525.67 | 18.09 | 526 |
| | phenethylamino)phenyl | amino)phenyl | Dimethylaminophenyl)-1- | | | | |
| | sulfanylphenyl]acetate | isocynate | phenethylureido] | | | | |
| | | | phenylsulfanyl}phenyl) | | | | |
| | | | acetic acid | | | | |
| 56b | Ethyl [4-(3- | benzyl | {4-[3-(3-Benzyl-1- | 3.5 | 490.67 | 87.45 | 491(prep) |
| | heptylaminophenylsulfanyl) | isocyanate | heptylureido)phenylsulfanyl]- | | | | |
| | phenyl]acetate | | phenyl}acetic acid | | | | |
| 57b | Ethyl [4-(3- | phenyl | {4-[3-(1-Heptyl-3- | 3.7 | 476.64 | 92.06 | 477(prep) |
| | heptylaminophenylsulfanyl) | isocyanate | phenylureido)phenylsulfanyl]- | | | | |
| | phenyl]acetate | | phenyl}acetic acid | | | | |
| 58b | Ethyl [4-(3- | heptyl | {4-[3-(1,3- | 15.6 | 498.73 | 81.78 | 499 |
| | heptylaminophenylsulfanyl) | isocyanate | Diheptylureido)phenylsulfanyl]- | | | | |
| | phenyl]acetate | | phenyl}acetic acid | | | | |
| 59b | Ethyl [4-(3- | phenethyl | {4-[3-(1-Heptyl-3- | 4.9 | 504.69 | 92.39 | 505(prep) |
| | heptylaminophenylsulfanyl) | isocyanate | phenethylureido)phenylsulfanyl] | | | | |
| | phenyl]acetate | | phenyl}acetic acid | | | | |
| 60b | Ethyl [4-(3- | 4-(trifluoromethyl) | (4-{3-[1-Heptyl-3-(4- | 3.6 | 544.64 | 79.05 | 545(prep) |
| | heptylaminophenylsulfanyl) | phenyl | trifluoromethylphenyl)- | | | | |
| | phenyl]acetate | isocyanate | ureido]phenylsulfanyl}phenyl) | | | | |
| | | | acetic acid | | | | |
| 61b | Ethyl [4-(3- | 4-methoxy | (4-{3-[1-Heptyl-3-(4- | 2.7 | 506.66 | 86.14 | 507(prep) |
| | heptylaminophenylsulfanyl) | phenyl | methoxyphenyl)ureido]- | | | | |
| | phenyl]acetate | isocyanate | phenylsulfanyl}phenyl)acetic | | | | |
| | | | acid | | | | |
| 62b | Ethyl [4-(3- | 1-adamantyl | {4-[3-(3-Adamantan-1-yl-1- | 11.2 | 534.76 | 35 | 535 |
| | heptylaminophenylsulfanyl) | isocyanate | heptylureido)- | | | | |
| | phenyl]acetate | | phenylsulfanyl]phenyl}acetic | | | | |
| | | | acid | | | | |
| 63b | Ethyl [4-(3- | 2-phenoxyphenyl | (4-{3-[1-Heptyl-3-(2- | 8.7 | 568.74 | 88.55 | 569 |
| | heptylaminophenylsulfanyl) | isocyanate | phenoxyphenyl)ureido]- | | | | |
| | phenyl]acetate | | phenylsulfanyl}phenyl)acetic | | | | |
| | | | acid | | | | |
| 64b | Ethyl [4-(3- | allyl | {4-[3-(3-Allyl-1- | 3.7 | 440.61 | 85.5 | 441 |
| | heptylaminophenylsulfanyl) | isocyanate | heptylureido)phenylsulfanyl]- | | | | |
| | phenyl]acetate | | phenyl}acetic acid | | | | |
| 65b | Ethyl [4-(3- | cyclohexyl | {4-[3-(3-Cyclohexyl-1- | 17.1 | 482.69 | 82.19 | 483 |
| | heptylaminophenylsulfanyl) | isocyanate | heptylureido)phenylsulfanyl] | | | | |
| | phenyl]acetate | | phenyl}acetic acid | | | | |
| 66b | Ethyl [4-(3- | 2-nitrophenyl | (4-{3-[1-Heptyl-3-(2- | 10.8 | 521.64 | 91 | 522 |
| | heptylaminophenylsulfanyl) | isocyanate | nitrophenyl)ureido]- | | | | |
| | phenyl]acetate | | phenylsulfanyl}phenyl)acetic | | | | |
| | | | acid | | | | |
| 67b | Ethyl [4-(3- | hexyl | {4-[3-(1-Heptyl-3- | 16.6 | 484.70 | 83.45 | 485 |
| | heptylaminophenylsulfanyl) | isocyanate | hexylureido)phenylsulfanyl] | | | | |
| | phenyl]acetate | | phenyl}acetic acid | | | | |
| 68b | Ethyl [4-(3- | 2-naphthyl | {4-[3-(1-Heptyl-3-naphthalen- | 14.4 | 526.70 | 74.3 | 527 |
| | heptylaminophenylsulfanyl) | isocyanate | 2-yl-ureido)- | | | | |
| | phenyl]acetate | | phenylsulfanyl]phenyl}acetic | | | | |
| | | | acid | | | | |
| 69b | Ethyl [4-(3- | 2- | (4-{3-[3-(2-Ethoxyphenyl)-1- | 18.6 | 520.69 | 88.51 | 521 |
| | heptylaminophenylsulfanyl) | ethoxyphenyl | heptylureido]- | | | | |
| | phenyl]acetate | isocyanate | phenylsulfanyl}phenyl)acetic | | | | |
| | | | acid | | | | |
| 70b | Ethyl [4-(3- | 4- | (4-{3-[3-(4-Butoxyphenyl)-1- | 25.5 | 548.75 | 56.02 | 549 |
| | heptylaminophenylsulfanyl) | butoxyphenyl | heptylureido]- | | | | |
| | phenyl]acetate | isocyanate | phenylsulfanyl}phenyl)acetic | | | | |
| | | | acid | | | | |
| 71b | Ethyl [4-(3- | pentyl | {4-[3-(1-Heptyl-3- | 16.2 | 470.68 | 89.45 | 471 |
| | heptylaminophenylsulfanyl) | isocyanate | pentylureido)phenylsulfanyl]- | | | | |
| | phenyl]acetate | | phenyl}acetic acid | | | | |
| 72b | Ethyl [4-(3- | n-butyl | {4-[3-(3-Butyl-1- | 11.4 | 456.65 | 91.58 | 457 |
| | heptylaminophenylsulfanyl) | isocyanate | heptylureido)phenylsulfanyl]- | | | | |
| | phenyl]acetate | | phenyl}acetic acid | | | | |
| 73b | Ethyl [4-(3- | 4-(dimethylamino) | (4-{3-[3-(4- | 12.5 | 519.71 | 47.52 | 520 |
| | heptylaminophenylsulfanyl) | phenyl | Dimethylaminophenyl)-1- | | | | |
| | phenyl]acetate | isocyanate | heptylureido]- | | | | |
| | | | phenylsulfanyl}phenyl)acetic | | | | |
| | | | acid | | | | |
| 74b | Ethyl [4-(3- | benzyl | {4-[3-(3-Benzyl-1- | 8.3 | 448.58 | 80.03 | 449 |
| | butylaminophenylsulfanyl) | isocyanate | butylureido)phenylsulfanyl]- | | | | |
| | phenyl]acetate | | phenyl}acetic acid | | | | |
| 75b | Ethyl [4-(3- | phenyl | {4-[3-(1-Butyl-3- | 5.5 | 434.56 | 81.38 | 435 |
| | butylaminophenylsulfanyl) | isocyanate | phenylureido)phenylsulfanyl]- | | | | |
| | phenyl]acetate | | phenyl}acetic acid | | | | |
| 76b | Ethyl [4-(3- | 2,3- | (4-{3-[1-Butyl-3-(2,3- | 2.6 | 503.45 | 82.37 | 503 |
| | butylaminophenylsulfanyl) | dichlorophenyl | dichlorophenyl)ureido]- | | | | |
| | phenyl]acetate | isocyanate | phenylsulfanyl}phenyl)acetic | | | | |
| | | | acid | | | | |
| 77b | Ethyl [4-(3- | heptyl | {4-[3-(1-Butyl-3- | 8.5 | 456.65 | 75.56 | 457 |
| | butylaminophenylsulfanyl) | isocyanate | heptylureido)phenylsulfanyl]- | | | | |
| | phenyl]acetate | | phenyl}acetic acid | | | | |
| 78b | Ethyl [4-(3- | phenethyl | {4-[3-(1-Butyl-3- | 8.3 | 462.61 | 79.07 | 463 |
| | butylaminophenylsulfanyl) | isocyanate | phenethylureido)phenylsulfanyl] | | | | |
| | phenyl]acetate | | phenyl}acetic acid | | | | |
| 79b | Ethyl [4-(3- | 4-(trifluoromethyl) | (4-{3-[1-Butyl-3-(4- | 6.2 | 502.55 | 58.9 | 503 |
| | butylaminophenylsulfanyl) | phenyl | trifluoromethylphenyl)ureido]- | | | | |
| | phenyl]acetate | isocyanate | phenylsulfanyl}phenyl)acetic | | | | |
| | | | acid | | | | |
| 80b | Ethyl [4-(3- | 4-methoxyphenyl | (4-{3-[1-Butyl-3-(4- | 6.8 | 464.58 | 76.76 | 465 |
| | butylaminophenylsulfanyl) | isocyanate | methoxyphenyl)ureido]- | | | | |
| | phenyl]acetate | | phenylsulfanyl}phenyl)acetic | | | | |
| | | | acid | | | | |
| 81b | Ethyl [4-(3- | 1-adamantyl | {4-[3-(3-Adamantan-1-yl-1- | 6.8 | 492.68 | 16.8 | 493 |
| | butylaminophenylsulfanyl) | isocyanate | butylureido)phenylsulfanyl] | | | | |
| | phenyl]acetate | | phenyl}acetic acid | | | | |
| 82b | Ethyl [4-(3- | 2-phenoxyphenyl | (4-{3-[1-Butyl-3-(2- | 8.4 | 526.65 | 84.34 | 527 |
| | butylaminophenylsulfanyl) | isocyanate | phenoxyphenyl)ureido]- | | | | |
| | phenyl]acetate | | phenylsulfanyl}phenyl)acetic | | | | |
| | | | acid | | | | |
| 83b | Ethyl [4-(3- | allyl | {4-[3-(3-Allyl-1- | 8.0 | 398.52 | 77.42 | 399 |
| | butylaminophenylsulfanyl) | isocyanate | butylureido)phenylsulfanyl]- | | | | |
| | phenyl]acetate | | phenyl}acetic acid | | | | |
| 84b | Ethyl [4-(3- | cyclohexyl | {4-[3-(1-Butyl-3- | 7.2 | 440.61 | 65.3 | 441 |
| | butylaminophenylsulfanyl) | isocyanate | cyclohexylureido)phenylsulfanyl] | | | | |
| | phenyl]acetate | | phenyl}acetic acid | | | | |
| 85b | Ethyl [4-(3- | 2-nitrophenyl | (4-{3-[1-Butyl-3-(2- | 7.7 | 479.55 | 91 | 480 |
| | butylaminophenylsulfanyl) | isocyanate | nitrophenyl)ureido]- | | | | |
| | phenyl]acetate | | phenylsulfanyl}phenyl)acetic | | | | |
| | | | acid | | | | |
| 86b | Ethyl [4-(3- | hexyl | {4-[3-(1-Butyl-3- | 7.3 | 442.62 | 85.74 | 443 |
| | butylaminophenylsulfanyl) | isocyanate | hexylureido)phenylsulfanyl] | | | | |
| | phenyl]acetate | | phenyl}acetic acid | | | | |
| 87b | Ethyl [4-(3- | 2-naphthyl | {4-[3-(1-Butyl-3-naphthalen- | 7.7 | 484.62 | 75.14 | 485 |
| | butylaminophenylsulfanyl) | isocyanate | 2-yl-ureido)- | | | | |
| | phenyl]acetate | | phenylsulfanyl]phenyl}acetic | | | | |
| | | | acid | | | | |
| 88b | Ethyl [4-(3- | 2- | (4-{3-[1-Butyl-3-(2- | 7.8 | 478.61 | 84.83 | 479 |
| | butylaminophenylsulfanyl) | ethoxyphenyl | ethoxyphenyl)ureido]phenylsulfanyl} | | | | |
| | phenyl]acetate | isocyanate | phenyl)acetic acid | | | | |
| 89b | Ethyl [4-(3- | 4- | (4-{3-[3-(4-Butoxyphenyl)-1- | 2.4 | 506.66 | 79.69 | 507 |
| | butylaminophenylsulfanyl) | butoxyphenyl | butylureido]phenylsulfanyl} | | | | |
| | phenyl]acetate | isocyanate | phenyl)acetic acid | | | | |
| 90b | Ethyl [4-(3- | pentyl | {4-[3-(1-Butyl-3- | 7.9 | 428.59 | 74.5 | 429 |
| | butylaminophenylsulfanyl) | isocyanate | pentylureido)phenylsulfanyl]- | | | | |
| | phenyl]acetate | | phenyl}acetic acid | | | | |
| 91b | Ethyl [4-(3- | n-butyl | {4-[3-(1,3- | 3.1 | 414.57 | 74.38 | 415 |
| | butylaminophenylsulfanyl) | isocyanate | Dibutylureido)phenylsulfanyl] | | | | |
| | phenyl]acetate | | phenyl)acetic acid | | | | |
| 92b | Ethyl {4-[4-(3- | benzyl | (4-{4-[3-Benzyl-1-(3- | 15.1 | 510.66 | 92.08 | 511 |
| | phenylpropylamino)phenylsulfanyl] | isocyanate | phenylpropyl)ureido]phenylsulfanyl} | | | | |
| | phenyl}acetate | | phenyl)acetic acid | | | | |
| 93b | Ethyl {4-[4-(3- | heptyl | (4-{4-[3-Heptyl-1-(3- | 3.4 | 518.72 | 70.96 | 519 |
| | phenylpropylamino)phenylsulfanyl] | isocyanate | phenylpropyl)ureido]phenylsulfanyl} | | | | |
| | phenyl}acetate | | phenyl)acetic acid | | | | |
| 94b | Ethyl {4-[4-(3- | phenethyl | (4-{4-[3-Phenethyl-1-(3- | 14.7 | 524.68 | 82.71 | 525 |
| | phenylpropylamino)phenylsulfanyl] | isocyanate | phenylpropyl)ureido]- | | | | |
| | phenyl}acetate | | phenylsulfanyl}phenyl)acetic | | | | |
| | | | acid | | | | |
| 95b | Ethyl {4-[4-(3- | 4-(trifluoromethyl) | (4-{4-[1-(3-Phenylpropyl)-3- | 7.1 | 564.63 | 22.07 | 565 |
| | phenylpropylamino)phenylsulfanyl] | phenyl | (4-trifluoromethylphenyl) | | | | |
| | phenyl}acetate | isocyanate | ureido] | | | | |
| | | | phenylsulfanyl}phenyl)acetic | | | | |
| | | | acid | | | | |
| 96b | Ethyl {4-[4-(3- | 2-phenoxyphenyl | (4-{4-[3-(2-Phenoxyphenyl)-1- | 19.3 | 588.73 | 76.89 | 589 |
| | phenylpropylamino)phenylsulfanyl] | isocyanate | (3-phenylpropyl) | | | | |
| | phenyl}acetate | | ureido]phenylsulfanyl} | | | | |
| | | | phenyl)acetic acid | | | | |
| 97b | Ethyl {4-[4-(3- | allyl | (4-{4-[3-Allyl-1-(3- | 11.5 | 460.60 | 88.2 | 461 |
| | phenylpropylamino)phenylsulfanyl] | isocyanate | phenylpropyl)ureido]phenylsulfanyl} | | | | |
| | phenyl}acetate | | phenyl)acetic acid | | | | |
| 98b | Ethyl {4-[4-(3- | cyclohexyl | (4-{4-[3-Cyclohexyl-1-(3- | 11.8 | 502.68 | 45.57 | 503 |
| | phenylpropylamino)phenylsulfanyl] | isocyanate | phenylpropyl)ureido]- | | | | |
| | phenyl}acetate | | phenylsulfanyl}phenyl)acetic | | | | |
| | | | acid | | | | |
| 99b | Ethyl {4-[4-(3- | 2-nitrophenyl | (4-{4-[3-(2-Nitrophenyl)-1-(3- | 30.1 | 541.63 | 77 | 542 |
| | phenylpropylamino)phenylsulfanyl] | isocyanate | phenylpropyl)- | | | | |
| | phenyl}acetate | | ureido]phenylsulfanyl}phenyl) | | | | |
| | | | acetic acid | | | | |
| 100b | MY 824.074.5 | hexyl | (4-{4-[3-Hexyl-1-(3- | 12.9 | 504.69 | 79.2 | 505 |
| | | isocyanate | phenylpropyl)ureido]phenylsulfanyl} | | | | |
| | | | phenyl)acetic acid | | | | |
| 101b | Ethyl {4-[4-(3- | 2-naphthyl | (4-{4-[3-Naphthalen-2-yl-1-(3- | 14.8 | 546.69 | 54.03 | 547 |
| | phenylpropylamino)phenylsulfanyl] | isocyanate | phenylpropyl) | | | | |
| | phenyl}acetate | | ureido]phenylsulfanyl} | | | | |
| | | | phenyl)acetic acid | | | | |
| 102b | Ethyl {4-[4-(3- | 2- | (4-{4-[3-(2-Ethoxyphenyl)-1-(3- | 34.3 | 540.68 | 70.75 | 541 |
| | phenylpropylamino)phenylsulfanyl] | ethoxyphenyl | phenylpropyl)- | | | | |
| | phenyl}acetate | isocyanate | ureido]phenylsulfanyl}phenyl) | | | | |
| | | | acetic acid | | | | |
| 103b | MY 824.074.5 | 4- | (4-{4-[3-(4-Butoxyphenyl)-1-(3- | 17.1 | 568.74 | 36.13 | 569 |
| | | butoxyphenyl | phenylpropyl)- | | | | |
| | | isocyanate | ureido]phenylsulfanyl}phenyl) | | | | |
| | | | acetic acid | | | | |
| 104b | Ethyl {4-[4-(3- | pentyl | (4-{4-[3-Pentyl-1-(3- | 14.1 | 490.67 | 73.12 | 491 |
| | phenylpropylamino)phenylsulfanyl] | isocyanate | phenylpropyl)ureido]- | | | | |
| | phenyl}acetate | | phenylsulfanyl}phenyl)acetic | | | | |
| | | | acid | | | | |
| 105b | Ethyl {4-[4-(3- | n-butyl | (4-{4-[3-Butyl-1-(3- | 11.9 | 476.64 | 94.96 | 477 |
| | phenylpropylamino)phenylsulfanyl] | isocyanate | phenylpropyl)ureido]- | | | | |
| | phenyl}acetate | | phenylsulfanyl}phenyl)acetic | | | | |
| | | | acid | | | | |
| 106b | Ethyl [4-(4- | benzyl | {4-[4-(3-Benzyl-1- | 12.8 | 496.63 | 92.47 | 497 |
| | phenethylaminophenylsulfanyl) | isocyanate | phenethylureido)phenylsulfanyl] | | | | |
| | phenyl]acetate | | phenyl}acetic acid | | | | |
| 107b | Ethyl [4-(4- | phenyl | {4-[4-(1-Phenethyl-3- | 11.7 | 482.60 | 75.58 | 483 |
| | phenethylaminophenylsulfanyl) | isocyanate | phenylureido)phenylsulfanyl] | | | | |
| | phenyl]acetate | | phenyl}acetic acid | | | | |
| 108b | Ethyl [4-(4- | 2,3- | (4-{4-[3-(2,3-Dichlorophenyl)- | 17.1 | 551.49 | 24.78 | 551 |
| | phenethylaminophenylsulfanyl) | dichlorophenyl | 1-phenethylureido]- | | | | |
| | phenyl]acetate | isocyanate | phenylsulfanyl}phenyl)acetic | | | | |
| | | | acid | | | | |
| 109b | Ethyl [4-(4- | heptyl | {4-[4-(3-Heptyl-1- | 20.5 | 504.69 | 82.55 | 505 |
| | phenethylaminophenysulfanyl) | isocyanate | phenethylureido)phenylsulfanyl] | | | | |
| | phenyl]acetate | | phenyl}acetic acid | | | | |
| 110b | Ethyl [4-(4- | phenethyl | {4-[4-(1,3- | 12.4 | 510.66 | 83.74 | 511 |
| | phenethylaminophenylsulfanyl) | isocyanate | Diphenethylureido)phenylsulfanyl] | | | | |
| | phenyl]acetate | | phenyl}acetic acid | | | | |
| 111b | Ethyl [4-(4- | 4-(trifluoro | (4-{4-[1-Phenethyl-3-(4- | 24.4 | 550.60 | 20.11 | 551 |
| | phenethylaminophenylsulfanyl) | methyl)phenyl | trifluoromethyl- | | | | |
| | phenyl]acetate | isocyanate | phenyl)ureido]phenylsulfanyl} | | | | |
| | | | phenyl)acetic acid | | | | |
| 112b | Ethyl [4-(4- | 4-methoxy | (4-{4-(3-Methoxyphenyl)-1- | 13.5 | 512.63 | 96.49 | 513 |
| | phenethylaminophenylsulfanyl) | phenyl | phenethylureido]- | | | | |
| | phenyl]acetate | isocyanate | phenylsulfanyl}phenyl)acetic | | | | |
| | | | acid | | | | |
| 113b | Ethyl [4-(4- | 1-adamantyl | {4-[4-(3-Adamantan-1-yl-1- | 12.2 | 540.73 | 50.4 | 541 |
| | phenethylaminophenylsulfanyl) | isocyanate | phenethylureido)- | | | | |
| | phenyl]acetate | | phenylsulfanyl]phenyl}acetic | | | | |
| | | | acid | | | | |
| 114b | Ethyl [4-(4- | 2-phenoxy | (4-{4-[1-Phenethyl-3-(2- | 13.2 | 574.70 | 86.26 | 575 |
| | phenethylaminophenylsulfanyl) | phenyl | phenoxyphenyl)ureido]- | | | | |
| | phenyl]acetate | isocyanate | phenylsulfanyl}phenyl)acetic | | | | |
| | | | acid | | | | |
| 115b | Ethyl [4-(4- | allyl | {4-[4-(3-Allyl-1- | 8.3 | 446.57 | 83.01 | 447 |
| | phenethylaminophenylsulfanyl) | isocyanate | phenethylureido)phenylsulfanyl] | | | | |
| | phenyl]acetate | | phenyl}acetic acid | | | | |
| 116b | Ethyl [4-(4- | cyclohexyl | {4-[4-(3-Cyclohexyl-1- | 9.3 | 488.65 | 77.7 | 489 |
| | phenethylaminophenylsulfanyl) | isocyanate | phenethylureido)- | | | | |
| | phenyl]acetate | | phenylsulfanylphenyl}acetic | | | | |
| | | | acid | | | | |
| 117b | Ethyl [4-(4- | 2-nitrophenyl | (4-{4-[3-(2-Nitrophenyl)-1- | 11.9 | 527.60 | 88 | 528 |
| | phenethylaminophenylsulfanyl) | isocyanate | phenethylureido]- | | | | |
| | phenyl]acetate | | phenylsulfanyl}phenyl)acetic | | | | |
| | | | acid | | | | |
| 118b | Ethyl [4-(4- | hexyl | {4-[4-(3-Hexyl-1- | 10.9 | 490.67 | 71.58 | 491 |
| | phenethylaminophenylsulfanyl) | isocyanate | phenethylureido)phenylsulfanyl] | | | | |
| | phenyl]acetate | | phenyl}acetic acid | | | | |
| 119b | Ethyl [4-(4- | 2-naphthyl | {4-[4-(3-Naphthalen-2-yl-1- | 7.9 | 532.66 | 60.52 | 533 |
| | phenethylaminophenylsulfanyl) | isocyanate | phenethylureido)- | | | | |
| | phenyl]acetate | | phenylsulfanyl]phenyl}acetic | | | | |
| | | | acid | | | | |
| 120b | Ethyl [4-(4- | 2- | (4-{4-[3-(2-Ethoxyphenyl)-1- | 16.2 | 526.65 | 82.87 | 527 |
| | phenethylaminophenylsulfanyl) | ethoxyphenyl | phenethylureido]- | | | | |
| | phenyl]acetate | isocyanate | phenylsulfanyl}phenyl)acetic | | | | |
| | | | acid | | | | |
| 121b | Ethyl [4-(4- | 4- | (4-{4-[3-(4-Butoxyphenyl)-1- | 20.6 | 554.71 | 38.53 | 555 |
| | phenethylaminophenylsulfanyl) | butoxyphenyl | phenethylureido]- | | | | |
| | phenyl]acetate | isocyanate | phenylsulfanyl}phenyl)acetic | | | | |
| | | | acid | | | | |
| 122b | Ethyl [4-(4- | pentyl | {4-[4-(3-Pentyl-1- | 3.8 | 476.64 | 81 | 477 |
| | phenethylaminophenylsulfanyl) | isocyanate | phenethylureido)phenylsulfanyl] | | | | |
| | phenyl]acetate | | phenyl}acetic acid | | | | |
| 123b | Ethyl [4-(4- | n-butyl | {4-[4-(3-Butyl-1- | 10.9 | 462.61 | 79.59 | 463 |
| | phenethylaminophenylsulfanyl) | isocyanate | phenethylureido)phenylsulfanyl] | | | | |
| | phenyl]acetate | | phenyl}acetic acid | | | | |
| 124b | Ethyl [4-(4- | benzyl | {4-[4-(3-Benzyl-1- | 28.1 | 490.67 | 85.96 | 491 |
| | heptylaminophenylsulfanyl) | isocyanate | heptylureido)phenylsulfanyl]- | | | | |
| | phenyl]acetate | | phenyl}acetic acid | | | | |
| 125b | Ethyl [4-(4- | phenyl | {4-[4-(1-heptyl-3- | 15.0 | 476.64 | 96.78 | 477 |
| | heptylaminophenylsulfanyl) | isocyanate | phenylureido)phenylsulfanyl]- | | | | |
| | phenyl]acetate | | phenyl}acetic acid | | | | |
| 126b | Ethyl [4-(4- | 2,3- | (4-{4-[3-(2,3-Dichlorophenyl)- | 16.9 | 545.53 | 43 | 546 |
| | heptylaminophenylsulfanyl) | dichlorophenyl | 1-heptylureido]- | | | | |
| | phenyl]acetate | isocyanate | phenylsulfanyl}phenyl)acetic | | | | |
| | | | acid | | | | |
| 127b | Ethyl [4-(4- | heptyl | {4-[4-(1,3- | 22.3 | 498.73 | 85.48 | 499 |
| | heptylaminophenylsulfanyl) | isocyanate | Diheptylureido)phenylsulfanyl] | | | | |
| | phenyl]acetate | | phenyl}acetic acid | | | | |
| 128b | Ethyl [4-(4- | phenethyl | {4-[4-(1-Heptyl-3- | 18.6 | 504.69 | 98.29 | 505 |
| | heptylaminophenylsulfanyl) | isocyanate | phenethylureido)phenylsulfanyl] | | | | |
| | phenyl]acetate | | phenyl}acetic acid | | | | |
| 129b | Ethyl [4-(4- | 4-(trifluoro | (4-{4-[1-Heptyl-3-(4- | 3.8 | 544.64 | 75.16 | 545(prep) |
| | heptylaminophenylsulfanyl) | methyl)phenyl | trifluoromethyl- | | | | |
| | phenyl]acetate | isocyanate | phenyl)ureido]phenylsulfanyl} | | | | |
| | | | phenyl)acetic acid | | | | |
| 130b | Ethyl [4-(4- | 4-methoxy | (4-{4-[1-Heptyl-3-(4- | 18.6 | 506.66 | 92.79 | 507 |
| | heptylaminophenylsulfanyl) | phenyl | methoxyphenyl)ureido]phenylsulfanyl} | | | | |
| | phenyl]acetate | isocyanate | phenyl)acetic acid | | | | |
| 131b | Ethyl [4-(4- | 1-adamantyl | {4-[4-(3-Adamantan-1-yl-1- | 10.3 | 534.76 | 50.19 | 535 |
| | heptylaminophenylsulfanyl) | isocyanate | heptylureido)- | | | | |
| | phenyl]acetate | | phenylsulfanyl]phenyl}acetic | | | | |
| | | | acid | | | | |
| 132b | Ethyl [4-(4- | 2-phenoxy | (4-{4-[1-Heptyl-3-(2- | 5.1 | 568.74 | 92.79 | 569 |
| | heptylaminophenylsulfanyl) | phenyl | phenoxyphenyl)ureido]- | | | | |
| | phenyl]acetate | isocyanate | phenylsulfanyl}phenyl)acetic | | | | |
| | | | acid | | | | |
| 133b | Ethyl [4-(4- | allyl | {4-[4-(3-Allyl-1- | 10.0 | 440.61 | 86.18 | 441 |
| | heptylaminophenylsulfanyl) | isocyanate | heptylureido)phenylsulfanyl]- | | | | |
| | phenyl]acetate | | phenyl}acetic acid | | | | |
| 134b | Ethyl [4-(4- | cyclohexyl | {4-[4-(3-Cyclohexyl-1- | 26.6 | 482.69 | 83.74 | 483 |
| | heptylaminophenylsulfanyl) | isocyanate | heptylureido)phenylsulfanyl] | | | | |
| | phenyl]acetate | | phenyl}acetic acid | | | | |
| 135b | Ethyl [4-(4- | 2-nitrophenyl | (4-{4-[1-Heptyl-3-(2- | 12.4 | 521.64 | 92 | 522 |
| | heptylaminophenylsulfanyl) | isocyanate | nitrophenyl)ureido]phenylsulfanyl} | | | | |
| | phenyl]acetate | | phenyl)acetic acid | | | | |
| 136b | Ethyl [4-(4- | hexyl | {4-[4-(1-Heptyl-3- | 13.9 | 484.70 | 94.84 | 485 |
| | heptylaminophenylsulfanyl) | isocyanate | hexylureido)phenylsulfanyl]- | | | | |
| | phenyl]acetate | | phenyl}acetic acid | | | | |
| 137b | Ethyl [4-(4- | 2-naphthyl | {4-[4-(1-Heptyl-3-naphthalen- | 15.2 | 526.70 | 77.28 | 527 |
| | heptylaminophenylsulfanyl) | isocyanate | 2-yl-ureido)- | | | | |
| | phenyl]acetate | | phenylsulfanyl]phenyl}acetic | | | | |
| | | | acid | | | | |
| 138b | Ethyl [4-(4- | 2- | (4-{4-[3-(2-Ethoxyphenyl)-1- | 20.8 | 520.69 | 83.86 | 521 |
| | heptylaminophenylsulfanyl) | ethoxyphenyl | heptylureido]- | | | | |
| | phenyl]acetate | isocyanate | phenylsulfanyl}phenyl)acetic | | | | |
| | | | acid | | | | |
| 139b | Ethyl [4-(4- | 4- | (4-{4-[3-(4-Butoxyphenyl)-1- | 28.8 | 548.75 | 31.13 | 549 |
| | heptylaminophenylsulfanyl) | butoxyphenyl | heptylureido]phenylsulfanyl} | | | | |
| | phenyl]acetate | isocyanate | phenyl)acetic acid | | | | |
| 140b | Ethyl [4-(4- | pentyl | {4-[4-(1-Heptyl-3- | 13.3 | 470.68 | 76.93 | 471 |
| | heptylaminophenylsulfanyl) | isocyanate | pentylureido)phenylsulfanyl]- | | | | |
| | phenyl]acetate | | phenyl}acetic acid | | | | |
| 141b | Ethyl [4-(4- | n-butyl | {4-[4-(3-Butyl-1- | 8.2 | 456.65 | 73.31 | 457 |
| | heptylaminophenylsulfanyl) | isocyanate | heptylureido)phenylsulfanyl]- | | | | |
| | phenyl]acetate | | phenyl}acetic acid | | | | |

Compounds 18a to 141a are the esters corresponding to the acids 18b to 141b obtained before the saponification step.

### Example 142

### Cross-Curve PPAR Transactivation Test

The activation of receptors with an agonist (activator) in HeLN cells leads to the expression of a reporter gene, luciferase, which, in the presence of a substrate, generates light. The modulation of the receptors is measured as quantity of luminescence produced after incubating the cells in the presence of a reference agonist. The ligands will displace the agonist from its site. The measurement of the activity is performed by quantification of the light produced. This measurement makes it possible to determine the modulatory activity of the compounds according to the invention by determining the constant which is the affinity of the molecule for the receptor. Since this value can fluctuate according to the basal activity and the expression of the receptor, it is called apparent Kd (KdApp in nM).

To determine this constant, “cross curves” for the product to be tested against a reference agonist are produced in a 96-well plate: 10 concentrations of the test product plus a concentration 0 are placed in a line, and 7 concentrations of the agonist plus one concentration 0 are placed in a column. This is 88 measurement points for 1 product and 1 receptor. The 8 remaining wells are used for repeatability controls.

In each well, the cells are in contact with a concentration of the product to be tested and a concentration of the reference agonist, 2-(4-{2-[3-(2,4-difluorophenyl)-1-heptylureido]ethyl}phenylsulfanyl)-2-methylpropionic acid for PPARα, {2-methyl-4-[4-methyl-2-(4-trifluoromethylphenyl)thiazol-5-ylmethylsulfanyl]phenoxy}acetic acid for PPARδ and 5-{4-[2-(methylpyridin-2-ylamino)ethoxy]benzyl}thiazolidine-2,4-dione for PPARγ. Measurements are also carried out for the controls total agonist with the same products.

The HeLN cell lines used are stable transfectants containing the plasmids ERE-pGlob-Luc-SV-Neo (reporter gene) and PPAR (α, δ, γ) Gal-hPPAR. These cells are inoculated into 96-well plates in an amount of 10 000 cells per well in 100 μl of DMEM medium free of phenol red and supplemented with 10% lipid-free calf serum. The plates are then incubated at 37° C., 7% CO₂ for 16 hours.

The various dilutions of the test products and of the reference ligand are added in an amount of 5 [I per well. The plates are then incubated for 18 hours at 37° C., 7% CO₂. The culture medium is removed by turning over and 100 μl of a 1:1 PBS/Luciferin mixture are added to each well. After 5 minutes, the plates are read by the luminescence reader.

These cross curves make it possible to determine the AC50 values (concentrations at which 50% activation is observed) for the reference ligand at various concentrations of test product. These AC50 values are used to calculate the Schild regression by plotting a straight line corresponding to the Schild equation (“quantitation in receptor pharmacology” Terry P. Kenakin, Receptors and Channels, 2001, 7, 371-385) which leads to Kd app values being obtained (in nM).

Transactivation results:

| | | | PPAR |
|---|---|---|---|
| | | | gamma |
| | PPAR alpha | PPAR delta | Kd app |
| Compounds | Kd app (nM) | Kd app (in nM) | (in nM) |
| Reference 1: 2-(4-{2- | 200 | n.a. | n.a. |
| [3-(2,4-Difluorophenyl)- | | | |
| 1-heptylureido]ethyl}phenyl- | | | |
| sulfanyl)-2-methyl propionic | | | |
| acid | | | |
| Reference 2: {2-Methyl- | n.a. | 10 | n.a. |
| 4-[4-methyl-2-(4- | | | |
| trifluoromethyl- | | | |
| phenyl)thiazol-5-ylmethyl- | | | |
| sulfanyl]phenoxy}acetic | | | |
| acid | | | |
| Reference 3: 5-{4-[2- | n.a. | n.a. | 30 |
| (Methylpyridin-2-ylamino)- | | | |
| ethoxy]benzyl}thiazolidine- | | | |
| 2,4-dione | | | |
| Example 30b | 8 000 | 120 | 2 000 |
| Example 116b | 250 | 120 | 500 |

n.a. means not active

### Example 143

### Compositions

Various specific formulations based on the compounds according to the invention are illustrated in this example.

A - ORAL ROUTE:
(a) 0.2 g tablet:

| | | |
|---|---|---|
| Compound of Example 2a | 0.001 | g |
| Starch | 0.114 | g |
| Bicalcium phosphate | 0.020 | g |
| Silica | 0.020 | g |
| Lactose | 0.030 | g |
| Talc | 0.010 | g |
| Magnesium stearate | 0.005 | g |

(b) Oral suspension in 5 ml vials:

| Compound of Example 7b | 0.001 | g |
|---|---|---|
| Glycerine | 0.500 | g |
| Sorbitol at 70% | 0.500 | g |
| Sodium saccharinate | 0.010 | g |
| Methyl para-hydroxybenzoate | 0.040 | g |
| Flavoring | qs | |
| Purified water qs | 5 | ml |

(c) 0.8 g tablet:

| | | |
|---|---|---|
| Compound of Example 45b | 0.500 | g |
| Pregelatinized starch | 0.100 | g |
| Microcrystalline cellulose | 0.115 | g |
| Lactose | 0.075 | g |
| Magnesium stearate | 0.010 | g |

(d) Oral suspension in 10 ml vials:

| Compound of Example 115a | 0.200 | g |
|---|---|---|
| Glycerine | 1.000 | g |
| Sorbitol at 70% | 1.000 | g |
| Sodium saccharinate | 0.010 | g |
| Methyl para-hydroxybenzoate | 0.080 | g |
| Flavoring | qs | |
| Purified water qs | 10 | ml |

B - TOPICAL ROUTE:
(a) Salve:

| | | |
|---|---|---|
| Compound of Example 76b | 0.020 | g |
| Isopropyl myristate | 81.700 | g |
| Fluid liquid paraffin | 9.100 | g |
| Silica (“Aerosil 200” sold by DEGUSSA) | 9.180 | g |

(b) Salve:

| | | |
|---|---|---|
| Compound of Example 95a | 0.300 | g |
| Petroleum jelly qs | 100 | g |

(c) Nonionic water-in-oil cream:

| | | |
|---|---|---|
| Compound of Example 46a | 0.100 | g |
| Mixture of emulsifying lanolin alcohols, waxes and | 39.900 | g |
| oils (“anhydrous eucerin” sold by BDF) | | |
| Methyl para-hydroxybenzoate | 0.075 | g |
| Propyl para-hydroxybenzoate | 0.075 | g |
| Sterile demineralized water qs | 100 | g |

(d) Lotion:

| | | |
|---|---|---|
| Compound of Example 57a | 0.100 | g |
| Polyethylene glycol (PEG 400) | 69.900 | g |
| Ethanol at 95% | 30.000 | g |

(e) Hydrophobic salve:

| | | |
|---|---|---|
| Compound of Example 21b | 0.300 | g |
| Isopropyl myristate | 36.400 | g |
| Silicone oil (“Rhodorsil 47 V 300” sold by | 36.400 | g |
| RHONE-POULENC) | | |
| Beeswax | 13.600 | g |
| Silicone oil (“Abil 300,000 cst” sold by | 100 | g |
| GOLDSCHMIDT) qs | | |

(f) Nonionic oil-in-water cream:

| | | |
|---|---|---|
| Compound of Example 19a | 1.000 | g |
| Cetyl alcohol | 4.000 | g |
| Glyceryl monostearate | 2.500 | g |
| PEG 50 stearate | 2.500 | g |
| Shea butter | 9.200 | g |
| Propylene glycol | 2.000 | g |
| Methyl para-hydroxybenzoate | 0.075 | g |
| Propyl para-hydroxybenzoate | 0.075 | g |
| Sterile demineralized water qs | 100 | g |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A compound having the following structural formula (I): 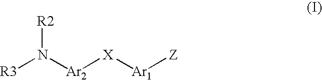
in which:
Ar₁ is an optionally substituted radical of one of the formulae (a)-(e): 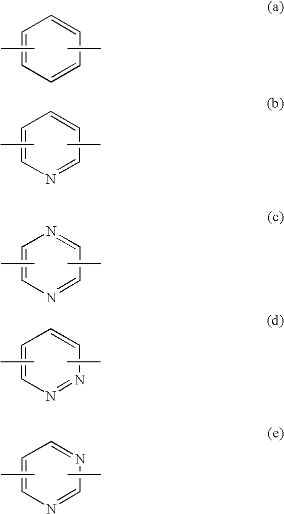
Z is the substituent: 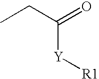
with the proviso that Z is at the para position with respect to X on the ring Ar₁;
R1 and Y are as defined below;
Ar₂ is an optionally substituted radical of one of the formulae (f)-(n): 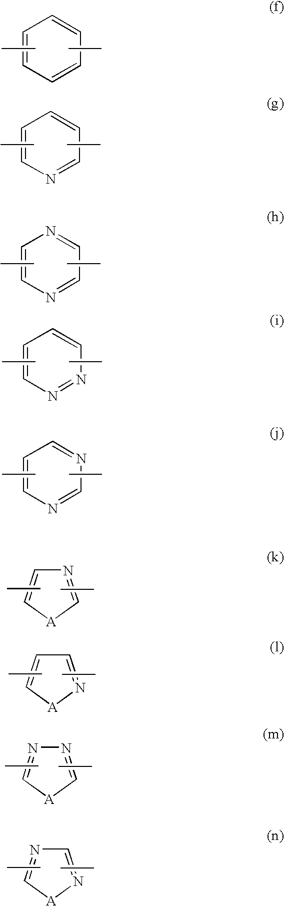
R1 is a hydrogen atom, an alkyl radical having from 1 to 12 carbon atoms, an aryl radical, a heteroaryl radical, an aralkyl radical, a polyether radical, a monohydroxyalkyl radical or a polyhydroxyalkyl radical;
R2 is a hydrogen atom, an alkyl radical having from 1 to 12 carbon atoms, an aryl radical, a heteroaryl radical, an aralkyl radical, a polyether radical, a monohydroxyalkyl radical or a polyhydroxyalkyl radical;
R3 is a hydrogen atom, an alkyl radical having from 1 to 12 carbon atoms, an aryl radical, a heteroaryl radical, an aralkyl radical, a polyether radical, a monohydroxyalkyl radical, a polyhydroxyalkyl radical, a radical COR5 or CSR5;
R5 is as defined below;
Y is an oxygen or sulfur atom, or the radical N—R4;
R4 is as defined below;
R4 is a hydrogen atom, an alkyl radical having from 1 to 12 carbon atoms, an aryl radical, a heteroaryl radical, a polyether radical, an aralkyl radical or together forms, with R1 and the nitrogen atom of Y, a heterocycle or a heteroaryl radical;
R5 is an aryl radical, a heteroaryl radical, an aralkyl radical, an alkyl radical having from 1 to 12 carbon atoms, a polyether radical, an alkoxy radical, a monohydroxyalkyl radical, a polyhydroxyalkyl radical, a radical R6-N—R7 or a radical O—R8;
R6, R7 and R8 are as defined below;
R6 and R7, which may be identical or different, are each a hydrogen atom, an alkyl radical having from 1 to 12 carbon atoms, a monohydroxyalkyl radical, a polyhydroxyalkyl radical, a polyether radical, an aryl radical, a heteroaryl radical, an aralkyl radical or together form a heterocycle;
R8 is a hydrogen atom, an alkyl radical having from 1 to 12 carbon atoms, a monohydroxyalkyl radical, a polyhydroxyalkyl radical, a polyether radical, an aryl radical, a heteroaryl radical or an aralkyl radical;
X is an S atom, a radical S═O, a radical O═S═O, an Se atom, an O atom, a radical N—R9, a radical C═O, a radical HO—C—R11 or a radical R10-C—R11;
R9, R10 and R11 are as defined below;
R9 is a hydrogen atom, a radical —COR12, an alkyl radical having from 1 to 12 carbon atoms, a polyether radical, an aryl radical or an aralkyl radical;
R12 is as defined below;
R10 and R11, which may be identical or different, are each a hydrogen atom, an alkyl radical having from 1 to 12 carbon atoms, an aryl radical, a heteroaryl radical, an aralkyl radical, a monohydroxyalkyl radical, a polyhydroxyalkyl radical, a polyether radical, an alkoxy radical, or R10 and R11 together form a ring member optionally interrupted by heteroatoms;
A is an S, O or Se atom or a radical N—R13;
R13 is as defined below;
R12 is an alkyl radical having from 1 to 12 carbon atoms;
R13 is a hydrogen atom, an alkyl radical having from 1 to 12 carbon atoms, an aryl radical, a heteroaryl radical, a polyether radical or an aralkyl radical;
and with the proviso when R2 is a hydrogen atom, R5 is different from an aryl radical and R3 is different from a hydrogen atom,
and the optical and geometric isomers of the said compounds of formula (I) and salts thereof.

2. The compound as defined by claim 1, wherein formula (I), Ar₁ is a radical (a).

3. The compound as defined by claim 1, wherein formula (I), Ar₁ is a radical (b).

4. The compound as defined by claim 1, wherein formula (I), Ar₁ is a radical (c).

5. The compound as defined by claim 1, wherein formula (I), Ar₁ is a radical (d).

6. The compound as defined by claim 1, wherein formula (I), Ar₁ is a radical (e).

7. The compound as defined by claim 1, wherein formula (I), Ar₂ is a radical (f).

8. The compound as defined by claim 1, wherein formula (I), Ar₂ is a radical (g).

9. The compound as defined by claim 1, wherein formula (I), Ar₂ is a radical (h).

10. The compound as defined by claim 1 wherein formula (I), Ar₂ is a radical (i).

11. The compound as defined by claim 1, wherein formula (I), Ar₂ is a radical (j).

12. The compound as defined by claim 1, wherein formula (I), Ar₂ is a radical (k).

13. The compound as defined by claim 1, wherein formula (I), Ar₂ is a radical (I).

14. The compound as defined by claim 1, wherein formula (I), Ar₂ is a radical (m).

15. The compound as defined by claim 1, wherein formula (I), Ar₂ is a radical (n).

16. The compound as defined by claim 1, wherein formula (I), Y is an oxygen atom.

17. The compound as defined by claim 1, wherein formula (I), Y is a sulfur atom.

18. The compound as defined by claim 1, wherein formula (I), Y is the radical N—R4.

19. The compound as defined by claim 1, wherein formula (I), X is an S atom, a radical S═O, a radical O═S═O, and Se atom, an O atom or a radical C═O.

20. The compound as defined by claim 1, wherein formula (I), X is a radical N—R9, a radical HO—C—R11 or a radical R10-C—R11.

21. The compound as defined by claim 1, wherein formula (I), R10 and R11 together form a dithianyl, dioxanyl, dithiolanyl, dioxolanyl or cyclopropanyl ring member.

22. An alkali or alkaline-earth metal, or zinc or organic amine salt of the compound as defined by claim 1.

23. The compound as defined by claim 1, comprising at least one alkyl radical having from 1 to 12 carbon atoms selected from the group consisting of hydrogenated or fluorinated, linear or cyclic, optionally branched radicals having 1 to 12 carbon atoms which may be interrupted by one or more heteroatoms.

24. The compound as defined by claim 1, comprising at least one 2-hydroxyethyl, 2-hydroxypropyl or 3-hydroxypropyl radical.

25. The compound as defined by claim 1, comprising at least one 2,3-dihydroxypropyl, 2,3,4-trihydroxybutyl or 2,3,4,5-tetrahydroxypentyl radical or the pentaerythritol residue.

26. The compound as defined by claim 1, comprising at least one methoxymethoxy, ethoxymethoxy or methoxyethoxymethoxy radical.

27. The compound as defined by claim 1, comprising at least one methoxy, ethoxy, isopropyloxy, tert-butoxy, hexyloxy, benzyloxy or phenoxy radical, optionally substituted with an alkyl radical having from 1 to 12 carbon atoms.

28. The compound as defined by claim 1, comprising at least one phenyl, biphenyl, cinnamyl or naphthyl radical which may be mono- or disubstituted with a halogen atom, a radical CF₃, an alkyl radical having from 1 to 12 carbon atoms, an alkoxy radical having from 1 to 7 carbon atoms, a nitro functional group, a polyether radical, an aryl radical, a benzoyl radical, an alkyl ester group, a carboxylic acid, a hydroxyl group optionally protected by an acetyl or benzoyl group or an amino functional group optionally protected by an acetyl or benzoyl group or optionally substituted with at least one alkyl radical having from 1 to 12 carbon atoms.

29. The compound as defined by claim 1, comprising at least one benzyl, phenethyl or naphthalen-2-ylmethyl radical which may be mono- or disubstituted with a halogen atom, a radical CF₃, an alkyl radical having from 1 to 12 carbon atoms, an alkoxy radical having from 1 to 7 carbon atoms, a nitro functional group, a polyether radical, an aryl radical, a benzoyl radical, an alkyl ester group, a carboxylic acid, a hydroxyl group optionally protected by an acetyl or benzoyl group or an amino functional group optionally protected by an acetyl or benzoyl group or optionally substituted with at least one alkyl radical having from 1 to 12 carbon atoms.

30. The compound as defined by claim 1, comprising at least one pyridyl, furyl, thienyl, isoxazolyl, oxadiazolyl, oxazolyl, isothiazolyl, quinazolinyl, benzothiadiazolyl, benzimidazole, indolyl or benzofuran radical, optionally substituted with at least one halogen, an alkyl radical having from 1 to 12 carbon atoms, an alkoxy radical having from 1 to 7 carbon atoms, an aryl radical, a nitro functional group, a polyether radical, a heteroaryl radical, a benzoyl radical, an alkyl ester group, a carboxylic acid, a hydroxyl group optionally protected by an acetyl or benzoyl group or an amino functional group optionally protected by an acetyl or benzoyl group or optionally substituted with at least one alkyl radical having from 1 to 12 carbon atoms.

31. The compound as defined by claim 1, comprising at least one morpholino, piperidino, piperazino, 2-oxopiperidin-1-yl or 2-oxopyrrolidin-1-yl radical optionally substituted with at least one alkyl radical having from 1 to 12 carbon atoms, an alkoxy radical having from 1 to 7 carbon atoms, an aryl radical, a nitro functional group, a polyether radical, a heteroaryl radical, a benzoyl radical, an alkyl ester group, a carboxylic acid, a hydroxyl group optionally protected by an acetyl or benzoyl group or an amino functional group optionally protected by an acetyl or benzoyl group or optionally substituted with at least one alkyl radical having from 1 to 12 carbon atoms.

32. The compound as defined by claim 1, selected from the group consisting of:
1b. Ethyl[4-(3-aminophenylsulfanyl)phenyl]acetate,
2. Ethyl[4-(4-aminophenylsulfanyl)phenyl]acetate,
3a. Ethyl{4-[3-(3-phenylpropylamino)phenylsulfanyl]phenyl}acetate,
3b. {4-[3-(3-Phenylpropylamino)phenylsulfanyl]phenyl}acetic acid,
4a. Ethyl(4-{3-[bis(3-phenylpropyl)amino]phenylsulfanyl}phenyl)acetate,
4b. (4-{3-[Bis(3-phenylpropyl)amino]phenylsuphanyl}phenyl)acetic acid,
5a. Ethyl[4-(3-Phenethylamino)phenylsulfanylphenyl]acetate,
5b. [4-(3-Phenethylamino)phenylsulfanylphenyl]acetic acid,
6a. Ethyl[4-(3-Diphenethylaminophenylsulfanyl)phenyl]acetate,
6b. [4-(3-Diphenethylaminophenylsulfanyl)phenyl]acetic acid,
7a. Ethyl[4-(3-heptylaminophenylsulfanyl)phenyl]acetate,
7b. [4-(3-Heptylaminophenylsulfanyl)phenyl]acetic acid,
8a. Ethyl[4-(3-diheptylaminophenylsulfanyl)phenyl]acetate,
8b. [4-(3-Diheptylaminophenylsulfanyl)phenyl]acetic acid,
9a. Ethyl[4-(3-butylaminophenylsulfanyl)phenyl]acetate,
9b. [4-(3-Butylaminophenylsulfanyl)phenyl]acetic acid,
10a. Ethyl[4-(3-dibutylaminophenylsulfanyl)phenyl]acetate,
10b. [4-(3-Dibutylaminophenylsulfanyl)phenyl]acetic acid,
11a. Ethyl{4-[4-(3-phenylpropylamino)phenylsulfanyl]phenyl}acetate,
11b. {4-[4-(3-Phenylpropylamino)phenylsulfanyl]phenyl}acetic acid,
12a. Ethyl(4-{4-[bis(3-phenylpropyl)amino]phenylsulfanyl}phenyl)acetate,
12b. (4-{4-[Bis(3-phenylpropyl)amino]phenylsulfanyl}phenyl)acetic acid,
13a. Ethyl[4-(4-phenethylaminophenylsulfanyl)phenyl]acetate,
13b. [4-(4-Phenethylaminophenylsulfanyl)phenyl]acetic acid,
14a. Ethyl[4-(4-diphenethylaminophenylsulfanyl)phenyl]acetate,
14b. [4-(4-Diphenethylaminophenylsulfanyl)phenyl]acetic acid,
15a. Ethyl[4-(4-heptylaminophenylsulfanyl)phenyl]acetate,
15b. [4-(4-Heptylaminophenylsulfanyl)phenyl]acetic acid,
16a. Ethyl[4-(4-diheptylaminophenylsulfanyl)phenyl]acetate,
16b. [4-(4-Diheptylaminophenylsulfanyl)phenyl]acetic acid,
17a. Ethyl[4-(4-dibutylaminophenylsulfanyl)phenyl]acetate,
17b. [4-(4-Dibutylaminophenylsulfanyl)phenyl]acetic acid,
18a. Ethyl(4-{3-[3-benzyl-1-(3-phenylpropyl)ureido]-phenylsulfanyl}phenyl)acetate,
18b. (4-{3-[3-Benzyl-1-(3-phenylpropyl)ureido]phenylsulfanyl}phenyl)acetic acid,
19a. Ethyl(4-{3-[3-phenyl-1-(3-phenylpropyl)ureido]phenylsulfanyl}phenyl)acetate,
19b. (4-{3-[3-Phenyl-1-(3-phenylpropyl)ureido]phenylsulfanyl}phenyl)acetic acid,
20a. Ethyl(4-{3-[3-(2,3-dichlorophenyl)-1-(3-phenylpropyl)ureido]phenylsulfanyl}phenyl)acetate,
20b. (4-{3-[3-(2,3-Dichlorophenyl)-1-(3-phenylpropyl)ureido]phenylsulfanyl}phenyl)acetic acid,
21a. Ethyl(4-{3-[3-heptyl-1-(3-phenylpropyl)ureido]phenylsulfanyl}phenyl)acetate,
21 b. (4-{3-[3-Heptyl-1-(3-phenylpropyl)ureido]phenylsulfanyl}phenyl)acetic acid,
22a. Ethyl(4-{3-[3-phenethyl-1-(3-phenylpropyl)ureido]phenylsulfanyl}phenyl)acetate,
22b. (4-{3-[3-Phenethyl-1-(3-phenylpropyl)ureido]phenylsulfanyl}phenyl)acetic acid,
23a. Ethyl(4-{3-[1-(3-phenylpropyl)-3-(4-trifluoromethylphenyl)ureido]phenylsulfanyl}phenyl)acetate,
23b. (4-{3-[1-(3-Phenylpropyl)-3-(4-trifluoromethylphenyl)ureido]phenylsulfanyl}phenyl)acetic acid,
24a. Ethyl(4-{3-[3-(4-methoxyphenyl)-1-(3-phenylpropyl)ureido]phenylsulfanyl}phenyl)acetate,
24b. (4-{3-[3-(4-Methoxyphenyl)-1-(3-phenylpropyl)ureido]phenylsulfanyl}phenyl)acetic acid,
25a. Ethyl(4-{3-[3-adamantan-1-yl-1-(3-phenylpropyl)-ureido]phenylsulfanyl}phenyl)acetate,
25b. (4-{3-[3-Adamantan-1-yl-1-(3-phenylpropyl)ureido]-phenylsulfanyl}phenyl)acetic acid,
26a. Ethyl(4-{3-[3-(2-phenoxyphenyl)-1-(3-phenylpropyl)-ureido]phenylsulfanyl}phenyl)acetate,
26b. (4-{3-[3-(2-Phenoxyphenyl)-1-(3-phenylpropyl)-ureido]phenylsulfanyl}phenyl)acetic acid,
27a. Ethyl(4-{3-[3-allyl-1-(3-phenylpropyl)ureido]-phenylsulfanyl}phenyl)acetate,
27b. (4-{3-[3-Allyl-1-(3-phenylpropyl)ureido]phenylsulfanyl}phenyl)acetic acid,
28a. Ethyl(4-{3-[3-cyclohexyl-1-(3-phenylpropyl)ureido]-phenylsulfanyl}phenyl)acetate,
28b. (4-{3-[3-Cyclohexyl-1-(3-phenylpropyl)ureido]-phenylsulfanyl}phenyl)acetic acid,
29a. Ethyl(4-{3-[3-(2-nitrophenyl)-1-(3-phenylpropyl)-ureido]phenylsulfanyl}phenyl)acetate,
29b. (4-{3-[3-(2-Nitrophenyl)-1-(3-phenylpropyl)ureido]-phenylsulfanyl}phenyl)acetic acid,
30a. Ethyl(4-{3-[3-hexyl-1-(3-phenylpropyl)ureido]-phenylsulfanyl}phenyl)acetate,
30b. (4-{3-[3-Hexyl-1-(3-phenylpropyl)ureido]phenylsulfanyl}phenyl)acetic acid,
31a. Ethyl(4-{3-.[3-naphthalen-2-yl-1-(3-phenylpropyl)ureido]-phenylsulfanyl}phenyl)acetate,
31 b. (4-{3-[3-Naphthalen-2-yl-1-(3-phenylpropyl)ureido]-phenylsulfanyl}phenyl)acetic acid,
32a. Ethyl(4-{3-[3-(2-ethoxyphenyl)-1-(3-phenylpropyl)-ureido]phenylsulfanyl}phenyl)acetate,
32b. (4-{3-[3-(2-Ethoxyphenyl)-1-(3-phenylpropyl)ureido]-phenylsulfanyl}phenyl)acetic acid,
33a. Ethyl(4-{3-[3-(4-butoxyphenyl)-1-(3-phenylpropyl)-ureido]phenylsulfanyl}phenyl)acetate,
33b. (4-{3-[3-(4-Butoxyphenyl)-1-(3-phenylpropyl)-ureido]phenylsulfanyl}phenyl)acetic acid,
34a. Ethyl(4-{3-[3-pentyl-1-(3-phenylpropyl)ureido]-phenylsulfanyl}phenyl)acetate,
34b. (4-{3-[3-Pentyl-1-(3-phenylpropyl)ureido]phenylsulfanyl}phenyl)acetic acid,
35a. Ethyl(4-{3-[3-butyl-1-(3-phenylpropyl)ureido]-phenylsulfanyl)phenyl)acetate,
35b. (4-{3-[3-Butyl-1-(3-phenylpropyl)ureido]phenylsulfanyl}phenyl)acetic acid,
36a. Ethyl(4-{3-[3-(4-dimethylaminophenyl)-1-(3-phenylpropyl)ureido]phenylsulfanyl}phenyl)acetate,
36b. (4-{3-[3-(4-Dimethylaminophenyl)-1-(3-phenylpropyl)-ureido]phenylsulfanyl}phenyl)acetic acid,
37a. Ethyl{4-[3-(3-benzyl-1-phenethylureido)phenylsulfanyl]-phenyl}acetate,
37b. {4-[3-(3-Benzyl-1-phenethylureido)phenylsulfanyl]phenyl}acetic acid,
38a. Ethyl{4-[3-(1-phenethyl-3-phenylureido)phenylsulfanyl]-phenyl}acetate,
38b. (4-[3-(1-Phenethyl-3-phenylureido)phenylsulfanyl]phenyl}acetic acid,
39a. Ethyl(4-{3-[3-(2,3-dichlorophenyl)-1-phenethylureido]-phenylsulfanyl}phenyl)acetate,
39b. (4-{3-[3-(2,3-Dichlorophenyl)-1-phenethylureido]-phenylsulfanyl}phenyl)acetic acid,
40a. Ethyl{4-[3-(3-heptyl-1-phenethylureido)phenylsulfanyl]phenyl}acetate,
40b. {4-[3-(3-Heptyl-1-phenethylureido)phenylsulfanyl]phenyl}acetic acid,
41a. Ethyl{4-[3-(1,3-diphenethylureido)phenylsulfanyl]phenyl}acetate,
41 b. {4-[3-(1,3-Diphenethylureido)phenylsulfanyl]phenyl}acetic acid,
42a. Ethyl(4-{3-[1-phenethyl-3-(4-trifluoromethyl-phenyl)ureido]phenylsulfanyl}phenyl)acetate,
42b. (4-{3-[1-Phenethyl-3-(4-trifluoromethylphenyl)-ureido]phenylsulfanyl}phenyl)acetic acid,
43a. Ethyl(4-{3-[3-(4-methoxyphenyl)-1-phenethylureido]phenylsulfanyl}phenyl)acetate,
43b. (4-{3-[3-(4-Methoxyphenyl)-1-phenethylureido]-phenylsulfanyl}phenyl)acetic acid,
44a. Ethyl{4-[3-(3-adamantan-1-yl-1-phenethylureido)-phenylsulfanyl]phenyl}acetate,
44b. {4-[3-(3-Adamantan-1-yl-1-phenethylureido)phenyl-sulfanyl]phenyl}acetic acid,
45a. Ethyl(4-{3-[1-phenethyl-3-(2-phenoxyphenyl)-ureido]phenylsulfanyl}phenyl)acetate,
45b. (4-{3-[1-Phenethyl-3-(2-phenoxyphenyl)ureido]-phenylsulfanyl}phenyl)acetic acid,
46a. Ethyl{4-[3-(3-allyl-1-phenethylureido)phenylsulfanyl]phenyl}acetate,
46b. {4-[3-(3-Allyl-1-phenethylureido)phenylsulfanyl]phenyl}acetic acid,
47a. Ethyl{4-[3-(3-cyclohexyl-1-phenethylureido)-phenylsulfanyl]phenyl}acetate,
47b. {4-[3-(3-Cyclohexyl-1-phenethylureido)phenylsulfanyl]phenyl}acetic acid,
48a. Ethyl(4-{3-[3-(2-nitrophenyl)-1-phenethylureido]-phenylsulfanyl}phenyl)acetate,
48b. (4-{3-[3-(2-Nitrophenyl)-1-phenethylureido]phenyl-sulfanyl}phenyl)acetic acid,
49a. Ethyl{4-[3-(3-hexyl-1-phenethylureido)phenylsulfanyl]phenyl}acetate,
49b. {4-[3-(3-Hexyl-1-phenethylureido)phenylsulfanyl]phenyl}acetic acid,
50a. Ethyl{4-[3-(3-naphthalen-2-yl-1-phenethylureido)-phenylsulfanyl]phenyl}acetate,
50b. {4-[3-(3-Naphthalen-2-yl-1-phenethylureido)phenyl-sulfanyl]phenyl}acetic acid,
51a. Ethyl(4-{3-[3-(2-ethoxyphenyl)-1-phenethylureido]phenylsulfanyl}phenyl)acetate,
51b. (4-{3-[3-(2-Ethoxyphenyl)-1-phenethylureido]-phenylsulfanyl}phenyl)acetic acid,
52a. Ethyl(4-{3-[3-(4-butoxyphenyl)-1-phenethylureido]phenylsulfanyl}phenyl)acetate,
52b. (4-{3-[3-(4-Butoxyphenyl)-1-phenethylureido]-phenylsulfanyl}phenyl)acetic acid,
53a. Ethyl{4-[3-(3-pentyl-1-phenethylureido)phenylsulfanyl]phenyl}acetate,
53b. {4-[3-(3-Pentyl-1-phenethylureido)phenylsulfanyl]phenyl}acetic acid,
54a. Ethyl{4-[3-(3-butyl-1-phenethylureido)phenylsulfanyl]phenyl}acetate,
54b. {4-[3-(3-Butyl-1-phenethylureido)phenylsulfanyl]phenyl}acetic acid,
55a. Ethyl(4-{3-[3-(4-dimethylaminophenyl)-1-phenethylureido]phenylsulfanyl}phenyl)acetate,
55b. (4-{3-[3-(4-Dimethylaminophenyl)-1-phenethylureido]phenylsulfanyl}phenyl)acetic acid,
56a. Ethyl{4-[3-(3-benzyl-1-heptylureido)phenylsulfanyl]phenyl}acetate,
56b. {4-[3-(3-Benzyl-1-heptylureido)phenylsulfanyl]phenyl}acetic acid,
57a. Ethyl{4-[3-(1-heptyl-3-phenylureido)phenylsulfanyl]phenyl}acetate,
57b. {4-[3-(1-Heptyl-3-phenylureido)phenylsulfanyl]phenyl}acetic acid,
58a. Ethyl{4-[3-(1,3-diheptylureido)phenylsulfanyl]phenyl}acetate,
58b. {4-[3-(1,3-Diheptylureido)phenylsulfanyl]phenyl}acetic acid,
59a. Ethyl{4-[3-(1-heptyl-3-phenethylureido)phenylsulfanyl]phenyl}acetate,
59b. {4-[3-(1-Heptyl-3-phenethylureido)phenylsulfanyl]phenyl}acetic acid,
60a. Ethyl(4-{3-[1-heptyl-3-(4-trifluoromethylphenyl)-ureido]phenylsulfanyl}phenyl)acetate,
60b. (4-{3-[1-Heptyl-3-(4-trifluoromethylphenyl)-ureido]phenylsulfanyl}phenyl)acetic acid,
61a. Ethyl(4-{3-[1-heptyl-3-(4-methoxyphenyl)ureido]-phenylsulfanyl}phenyl)acetate,
61 b. (4-{3-[1-Heptyl-3-(4-methoxyphenyl)ureido]-phenylsulfanyl}phenyl)acetic acid,
62a. Ethyl{4-[3-(3-adamantan-1-yl-1-heptylureido)-phenylsulfanyl]phenyl}acetate,
62b. {4-[3-(3-Adamantan-1-yl-1-heptylureido)phenylsulfanyl]phenyl}acetic acid,
63a. Ethyl(4-{3-[1-heptyl-3-(2-phenoxyphenyl)ureido]-phenylsulfanyl}phenyl)acetate,
63b. (4-{3-[1-Heptyl-3-(2-phenoxyphenyl)ureido]-phenylsulfanyl}phenyl)acetic acid,
64a. Ethyl{4-[3-(3-allyl-1-heptylureido)phenylsulfanyl]phenyl}acetate,
64b. {4-[3-(3-Allyl-1-heptylureido)phenylsulfanyl]phenyl}acetic acid,
65a. Ethyl{4-[3-(3-cyclohexyl-1-heptylureido)phenyl-sulfanyl]phenyl}acetate,
65b. {4-[3-(3-Cyclohexyl-1-heptylureido)phenylsulfanyl]phenyl}acetic acid,
66a. Ethyl(4-{3-[1-heptyl-3-(2-nitrophenyl)ureido]-phenylsulfanyl}phenyl)acetate,
66b. (4-{3-[1-Heptyl-3-(2-nitrophenyl)ureido]phenylsulfanyl}phenyl)acetic acid,
67a. Ethyl{4-[3-(1-heptyl-3-hexylureido)phenylsulfanyl]phenyl}acetate,
67b. {4-[3-(1-Heptyl-3-hexylureido)phenylsulfanyl]phenyl}acetic acid,
68a. Ethyl{4-[3-(1-heptyl-3-naphthalen-2-ylureido)-phenylsulfanyl]phenyl}acetate,
68b. {4-[3-(1-Heptyl-3-naphthalen-2-ylureido)phenylsulfanyl]phenyl}acetic acid,
69a. Ethyl(4-{3-[3-(2-ethoxyphenyl)-1-heptylureido]-phenylsulfanyl}phenyl)acetate,
69b. (4-{3-[3-(2-Ethoxyphenyl)-1-heptylureido]phenylsulfanyl}phenyl)acetic acid,
70a. Ethyl(4-{3-[3-(4-butoxyphenyl)-1-heptylureido]-phenylsulfanyl}phenyl)acetate,
70b. (4-{3-[3-(4-Butoxyphenyl)-1-heptylureido]phenylsulfanyl}phenyl)acetic acid,
71a. Ethyl{4-[3-(1-heptyl-3-pentylureido)phenylsulfanyl]phenyl}acetate,
71b. {4-[3-(1-Heptyl-3-pentylureido)phenylsulfanyl]phenyl}acetic acid,
72a. Ethyl{4-[3-(3-butyl-1-heptylureido)phenylsulfanyl]phenyl}acetate,
72b. {4-[3-(3-Butyl-1-heptylureido)phenylsulfanyl]phenyl}acetic acid,
73a. Ethyl(4-{3-[3-(4-dimethylaminophenyl)-1-heptyl-ureido]phenylsulfanyl}phenyl)acetate,
73b. (4-{3-[3-(4-Dimethylaminophenyl)-1-heptylureido]-phenylsulfanyl}phenyl)acetic acid,
74a. Ethyl{4-[3-(3-benzyl-1-butylureido)phenylsulfanyl]phenyl}acetate,
74b. {4-[3-(3-Benzyl-1-butylureido)phenylsulfanyl]phenyl}acetic acid,
75a. Ethyl{4-[3-(1-butyl-3-phenylureido)phenylsulfanyl]phenyl}acetate,
75b. {4-[3-(1-Butyl-3-phenylureido)phenylsulfanyl]phenyl}acetic acid,
76a. Ethyl(4-{3-[1-butyl-3-(2,3-dichlorophenyl)-ureido]phenylsulfanyl}phenyl)acetate,
76b. (4-{3-[1-Butyl-3-(2,3-dichlorophenyl)ureido]-phenylsulfanyl}phenyl)acetic acid,
77a. Ethyl{4-[3-(1-butyl-3-heptylureido)phenylsulfanyl]phenyl}acetate,
77b. {4-[3-(1-Butyl-3-heptylureido)phenylsulfanyl]phenyl}acetic acid,
78a. Ethyl{4-[3-(1-butyl-3-phenethylureido)phenylsulfanyl]phenyl}acetate,
78b. {4-[3-(1-Butyl-3-phenethylureido)phenylsulfanyl]phenyl}acetic acid,
79a. Ethyl(4-{3-[1-butyl-3-(4-trifluoromethylphenyl)-ureido]phenylsulfanyl}phenyl)acetate,
79b. (4-{3-[1-Butyl-3-(4-trifluoromethylphenyl)ureido]-phenylsulfanyl}phenyl)acetic acid,
80a. Ethyl(4-{3-[1-butyl-3-(4-methoxyphenyl)ureido]-phenylsulfanyl}phenyl)acetate,
80b. (4-{3-[1-Butyl-3-(4-methoxyphenyl)ureido]phenylsulfanyl}phenyl)acetic acid,
81a. Ethyl{4-[3-(3-adamantan-1-yl-1-butylureido)-phenylsulfanyl]phenyl}acetate,
81 b. {4-[3-(3-Adamantan-1-yl-1-butylureido)phenylsulfanyl]phenyl}acetic acid,
82a. Ethyl(4-{3-[1-butyl-3-(2-phenoxyphenyl)ureido]-phenylsulfanyl}phenyl)acetate,
82b. (4-{3-[1-Butyl-3-(2-phenoxyphenyl)ureido]phenylsulfanyl}phenyl)acetic acid,
83a. Ethyl{4-[3-(3-allyl-1-butylureido)phenylsulfanyl]phenyl}acetate,
83b. {4-[3-(3-Allyl-1-butylureido)phenylsulfanyl]phenyl}acetic acid,
84a. Ethyl{4-[3-(1-butyl-3-cyclohexylureido)phenylsulfanyl]phenyl}acetate,
84b. {4-[3-(1-Butyl-3-cyclohexylureido)phenylsulfanyl]phenyl}acetic acid,
85a. Ethyl(4-{3-[1-butyl-3-(2-nitrophenyl)ureido]-phenylsulfanyl}phenyl)acetate,
85b. (4-{3-[1-Butyl-3-(2-nitrophenyl)ureido]phenylsulfanyl}phenyl)acetic acid,
86a. Ethyl{4-[3-(1-butyl-3-hexylureido)phenylsulfanyl]phenyl}acetate,
86b. {4-[3-(1-Butyl-3-hexylureido)phenylsulfanyl]phenyl}acetic acid,
87a. Ethyl{4-[3-(1-butyl-3-naphthalen-2-ylureido)-phenylsulfanyl]phenyl}acetate,
87b. {4-[3-(1-Butyl-3-naphthalen-2-ylureido)phenylsulfanyl]phenyl}acetic acid,
88a. Ethyl(4-{3-[1-butyl-3-(2-ethoxyphenyl)ureido]-phenylsulfanyl}phenyl)acetate,
88b. (4-{3-[1-Butyl-3-(2-ethoxyphenyl)ureido]phenylsulfanyl}phenyl)acetic acid,
89a. Ethyl(4-{3-[3-(4-butoxyphenyl)-1-butylureido]-phenylsulfanyl}phenyl)acetate,
89b. (4-{3-[3-(4-Butoxyphenyl)-1-butylureido]phenylsulfanyl}phenyl)acetic acid,
90a. Ethyl{4-[3-(1-butyl-3-pentylureido)phenylsulfanyl]phenyl}acetate,
90b. {4-[3-(1-Butyl-3-pentylureido)phenylsulfanyl]phenyl}acetic acid,
91a. Ethyl{4-[3-(1,3-dibutylureido)phenylsulfanyl]phenyl}acetate,
91b. {4-[3-(1,3-Dibutylureido)phenylsulfanyl]phenyl}acetic acid,
92a. Ethyl(4-{4-[3-benzyl-1-(3-phenylpropyl)ureido]-phenylsulfanyl}phenyl)acetate,
92b. (4-{4-[3-Benzyl-1-(3-phenylpropyl)ureido]phenylsulfanyl}phenyl)acetic acid,
93a. Ethyl(4-{4-[3-heptyl-1-(3-phenylpropyl)ureido]-phenylsulfanyl}phenyl)acetate,
93b. (4-{4-[3-Heptyl-1-(3-phenylpropyl)ureido]phenylsulfanyl}phenyl)acetic acid,
94a. Ethyl(4-{4-[3-phenethyl-1-(3-phenylpropyl)-ureido]phenylsulfanyl}phenyl)acetate,
94b. (4-{4-[3-Phenethyl-1-(3-phenylpropyl)ureido]-phenylsulfanyl}phenyl)acetic acid,
95a. Ethyl(4-{4-[1-(3-phenylpropyl)-3-(4-tri-fluoromethylphenyl)ureido]phenylsulfanyl}phenyl)acetate,
95b. (4-{4-[1-(3-Phenylpropyl)-3-(4-tri-fluoromethylphenyl)ureido]phenylsulfanyl}phenyl)acetic acid,
96a. Ethyl(4-{4-[3-(2-phenoxyphenyl)-1-(3-phenyl-propyl)ureido]phenylsulfanyl}phenyl)acetate,
96b. (4-{4-[3-(2-Phenoxyphenyl)-1-(3-phenyl-propyl)ureido]phenylsulfanyl}phenyl)acetic acid,
97a. Ethyl(4-{4-[3-allyl-1-(3-phenylpropyl)ureido]-phenylsulfanyl}phenyl)acetate,
97b. (4-{4-[3-Allyl-1-(3-phenylpropyl)ureido]phenylsulfanyl}phenyl)acetic acid,
98a. Ethyl(4-{4-β-cyclohexyl-1-(3-phenylpropyl)-ureido]phenylsulfanyl}phenyl)acetate,
98b. (4-{4-[3-Cyclohexyl-1-(3-phenylpropyl)ureido]-phenylsulfanyl}phenyl)acetic acid,
99a. Ethyl(4-{4-[3-(2-nitrophenyl)-1-(3-phenyl-propyl)ureido]phenylsulfanyl}phenyl)acetate,
99b. (4-{4-[3-(2-Nitrophenyl)-1-(3-phenylpropyl)-ureido]phenylsulfanyl}phenyl)acetic acid,
100a. Ethyl(4-{4-[3-hexyl-1-(3-phenylpropyl)-ureido]phenylsulfanyl}phenyl)acetate,
100b. (4-{4-[3-Hexyl-1-(3-phenylpropyl)ureido]phenylsulfanyl}phenyl)acetic acid,
101a. Ethyl(4-{4-[3-naphthalen-2-yl-1-(3-phenyl-propyl)ureido]phenylsulfanyl}phenyl)acetate,
101 b. (4-{4-[3-Naphthalen-2-yl-1-(3-phenyl-propyl)ureido]phenylsulfanyl}phenyl)acetic acid,
102a. Ethyl(4-{4-[3-(2-ethoxyphenyl)-1-(3-phenyl-propyl)ureido]phenylsulfanyl}phenyl)acetate,
102b. (4-{4-[3-(2-Ethoxyphenyl)-1-(3-phenylpropyl)-ureido]phenylsulfanyl}phenyl)acetic acid,
103a. Ethyl(4-{4-[3-(4-butoxyphenyl)-1-(3-phenyl-propyl)ureido]phenylsulfanyl}phenyl)acetate,
103b. (4-{4-[3-(4-Butoxyphenyl)-1-(3-phenylpropyl)-ureido]phenylsulfanyl}phenyl)acetic acid,
104a. Ethyl(4-{4-[3-pentyl-1-(3-phenylpropyl)-ureido]phenylsulfanyl}phenyl)acetate,
104b. (4-{4-[3-Pentyl-1-(3-phenylpropyl)ureido]-phenylsulfanyl}phenyl)acetic acid,
105a. Ethyl(4-{4-[3-butyl-1-(3-phenylpropyl)ureido]-phenylsulfanyl}phenyl)acetate,
105b. (4-{4-[3-Butyl-1-(3-phenylpropyl)ureido]phenylsulfanyl}phenyl)acetic acid,
106a. Ethyl{4-[4-(3-benzyl-1-phenethylureido)-phenylsulfanyl]phenyl}acetate,
106b. {4-[4-(3-Benzyl-1-phenethylureido)phenylsulfanyl]phenyl}acetic acid,
107a. Ethyl{4-[4-(1-phenethyl-3-phenylureido)phenyl-sulfanyl]phenyl}acetate,
107b. {4-[4-(1-Phenethyl-3-phenylureido)phenylsulfanyl]phenyl}acetic acid.
108a. Ethyl(4-{4-[3-(2,3-dichlorophenyl)-1-phenethylureido]phenylsulfanyl}phenyl)acetate,
108b. (4-{4-[3-(2,3-Dichlorophenyl)-1-phenethylureido]-phenylsulfanyl}phenyl)acetic acid,
109a. Ethyl{4-[4-(3-heptyl-1-phenethylureido)phenyl-sulfanyl]phenyl}acetate,
109b. {4-[4-(3-Heptyl-1-phenethylureido)phenylsulfanyl]phenyl}acetic acid,
110a. Ethyl{4-[4-(1,3-diphenethylureido)phenylsulfanyl]phenyl}acetate,
110b. {4-[4-(1,3-Diphenethylureido)phenylsulfanyl]phenyl}acetic acid,
11 la. Ethyl(4-{4-[1-phenethyl-3-(4-trifluoro-methylphenyl)ureido]phenylsulfanyl}phenyl)acetate,
111 b. (4-{4-[1-Phenethyl-3-(4-trifluoromethyl-phenyl)ureido]phenylsulfanyl}phenyl)acetic acid,
112a. Ethyl(4-{4-[3-(4-methoxyphenyl)-1-phenethylureido]phenylsulfanyl}phenyl)acetate,
112b. (4-{4-[3-(4-Methoxyphenyl)-1-phenethylureido]-phenylsulfanyl}phenyl)acetic acid,
113a. Ethyl{4-[4-(3-adamantan-1-yl-1-phenethyl-ureido)phenylsulfanyl]phenyl}acetate,
113b. {4-[4-(3-Adamantan-1-yl-1-phenethylureido)-phenylsulfanyl]phenyl}acetic acid,
114a. Ethyl(4-{4-[1-phenethyl-3-(2-phenoxyphenyl)-ureido]phenylsulfanyl}phenyl)acetate,
114b. (4-{4-[1-Phenethyl-3-(2-phenoxyphenyl)ureido]-phenylsulfanyl}phenyl)acetic acid,
115a. Ethyl{4-[4-(3-allyl-1-phenethylureido)phenylsulfanyl]phenyl}acetate,
115b. {4-[4-(3-Allyl-1-phenethylureido)phenylsulfanyl]phenyl}acetic acid,
116a. Ethyl{4-[4-(3-cyclohexyl-1-phenethylureido)-phenylsulfanyl]phenyl}acetate,
116b. {4-[4-(3-Cyclohexyl-1-phenethylureido)phenylsulfanyl]phenyl}acetic acid,
117a. Ethyl(4-{4-[3-(2-nitrophenyl)-1-phenethylureido]phenylsulfanyl}phenyl)acetate,
117b. (4-{4-[3-(2-Nitrophenyl)-1-phenethylureido]-phenylsulfanyl}phenyl)acetic acid,
118a. Ethyl{4-[4-(3-hexyl-1-phenethylureido)-phenylsulfanyl]phenyl}acetate,
118b. {4-[4-(3-Hexyl-1-phenethylureido)phenylsulfanyl]phenyl}acetic acid,
119a. Ethyl{4-[4-(3-naphthalen-2-yl-1-phenethyl-ureido)phenylsulfanyl]phenyl}acetate,
119b. {4-[4-(3-Naphthalen-2-yl-1-phenethylureido)-phenylsulfanyl]phenyl}acetic acid,
120a. Ethyl(4-{4-[3-(2-ethoxyphenyl)-1-phenethylureido]phenylsulfanyl}phenyl)acetate,
120b. (4-{4-[3-(2-Ethoxyphenyl)-1-phenethylureido]-phenylsulfanyl}phenyl)acetic acid,
121a. Ethyl(4-{4-[3-(4-butoxyphenyl)-1-phenethylureido]phenylsulfanyl}phenyl)acetate,
121b. (4-{4-[3-(4-Butoxyphenyl)-1-phenethylureido]-phenylsulfanyl}phenyl)acetic acid,
122a. Ethyl{4-[4-(3-pentyl-1-phenethylureido)phenyl-sulfanyl]phenyl}acetate,
122b. {4-[4-(3-Pentyl-1-phenethylureido)phenylsulfanyl]phenyl}acetic acid,
123a. Ethyl{4-[4-(3-butyl-1-phenethylureido)phenylsulfanyl]phenyl}acetate,
123b. {4-[4-(3-Butyl-1-phenethylureido)phenylsulfanyl]phenyl}acetic acid,
124a. Ethyl{4-[4-(3-benzyl-1-heptylureido)phenylsulfanyl]phenyl}acetate,
124b. {4-[4-(3-Benzyl-1-heptylureido)phenylsulfanyl]phenyl}acetic acid,
125a. Ethyl{4-[4-(1-heptyl-3-phenylureido)phenylsulfanyl]phenyl}acetate,
125b. {4-[4-(1-Heptyl-3-phenylureido)phenylsulfanyl]phenyl}acetic acid,
126a. Ethyl(4-{4-[3-(2,3-dichlorophenyl)-1-heptyl-ureido]phenylsulfanyl}phenyl)acetate,
126b. (4-{4-[3-(2,3-Dichlorophenyl)-1-heptylureido]-phenylsulfanyl}phenyl)acetic acid,
127a. Ethyl{4-[4-(1,3-diheptylureido)phenylsulfanyl]phenyl}acetate,
127b. {4-[4-(1,3-Diheptylureido)phenylsulfanyl]phenyl}acetic acid,
128a. Ethyl{4-[4-(1-heptyl-3-phenethylureido)phenyl-sulfanyl]phenyl}acetate,
128b. {4-[4-(1-Heptyl-3-phenethylureido)phenylsulfanyl]phenyl}acetic acid,
129a. Ethyl(4-{4-[1-heptyl-3-(4-trifluoro-methylphenyl)ureido]phenylsulfanyl}phenyl)acetate,
129b. (4-{4-[1-Heptyl-3-(4-trifluoromethyl-phenyl)ureido]phenylsulfanyl}phenyl)acetic acid,
130a. Ethyl(4-{4-[1-heptyl-3-(4-methoxyphenyl)ureido]-phenylsulfanyl}phenyl)acetate,
130b. (4-{4-[1-Heptyl-3-(4-methoxyphenyl)ureido]phenyl-sulfanyl}phenyl)acetic acid,
131a. Ethyl{4-[4-(3-adamantan-1-yl-1-heptylureido)-phenylsulfanyl]phenyl}acetate,
131b. {4-[4-(3-Adamantan-1-yl-1-heptylureido)phenylsulfanyl]phenyl}acetic acid,
132a. Ethyl(4-{4-[1-heptyl-3-(2-phenoxyphenyl)-ureido]phenylsulfanyl}phenyl)acetate,
132b. (4-{4-[1-Heptyl-3-(2-phenoxyphenyl)ureido]-phenylsulfanyl}phenyl)acetic acid,
133a. Ethyl{4-[4-(3-allyl-1-heptylureido)phenylsulfanyl]phenyl}acetate,
133b. {4-[4-(3-Allyl-1-heptylureido)phenylsulfanyl]phenyl}acetic acid,
134a. Ethyl{4-[4-(3-cyclohexyl-1-heptylureido)phenyl-sulfanyl]phenyl}acetate,
134b. {4-[4-(3-Cyclohexyl-1-heptylureido)phenylsulfanyl]phenyl}acetic acid,
135a. Ethyl(4-{4-[1-heptyl-3-(2-nitrophenyl)ureido]-phenylsulfanyl}phenyl)acetate,
135b. (4-{4-[1-Heptyl-3-(2-nitrophenyl)ureido]phenylsulfanyl}phenyl)acetic acid,
136a. Ethyl{4-[4-(1-heptyl-3-hexylureido)phenylsulfanyl]phenyl}acetate,
136b. {4-[4-(1-Heptyl-3-hexylureido)phenylsulfanyl]phenyl}acetic acid,
137a. Ethyl{4-[4-(1-heptyl-3-naphthalen-2-ylureido)-phenylsulfanyl]phenyl}acetate,
137b. {4-[4-(1-Heptyl-3-naphthalen-2-ylureido)phenylsulfanyl]phenyl}acetic acid,
138a. Ethyl(4-{4-[3-(2-ethoxyphenyl)-1-heptylureido]-phenylsulfanyl}phenyl)acetate,
138b. (4-{4-[3-(2-Ethoxyphenyl)-1-heptylureido]-phenylsulfanyl}phenyl)acetic acid,
139a. Ethyl(4-{4-[3-(4-butoxyphenyl)-1-heptylureido]-phenylsulfanyl}phenyl)acetate,
139b. (4-{4-[3-(4-Butoxyphenyl)-1-heptylureido]phenyl-sulfanyl}phenyl)acetic acid,
140a. Ethyl{4-[4-(1-heptyl-3-pentylureido)phenylsulfanyl]phenyl}acetate,
140b. {4-[4-(1-Heptyl-3-pentylureido)phenylsulfanyl]phenyl}acetic acid,
141a. Ethyl{4-[4-(3-butyl-1-heptylureido)phenylsulfanyl]phenyl}acetate,
141b. {4-[4-(3-Butyl-1-heptylureido)phenylsulfanyl]phenyl}acetic acid, and mixtures thereof.

33. A cosmetic/pharmaceutical composition comprising a thus effective amount of at least one compound (I) as defined by claim 1, formulated into a physiologically acceptable carrier therefor.

34. The cosmetic/pharmaceutical composition as defined by claim 33, said at least one compound (I) comprising from 0.001% to 3% by weight thereof.

35. The cosmetic/pharmaceutical composition as defined by claim 33, said at least one compound (I) comprising from 0.001% to 10% by weight thereof.

36. The cosmetic/pharmaceutical composition as defined by claim 33, said at least one compound (I) comprising from 0.01% to 1% by weight thereof.

37. A regime or regimen for regulating and/or restoring skin lipid metabolism, comprising administering to a mammalian organism in need of such treatment, a thus effective amount of the cosmetic/pharmaceutical composition as defined by claim 33.

38. A regime or regimen for modulating PPAR-type receptors, comprising administering to a mammalian organism in need of such treatment, a thus effective amount of the cosmetic/pharmaceutical composition as defined by claim 33.

39. A regime or regimen for the treatment:
of dermatological conditions linked to a cicatrization disorder related to cell differentiation and proliferation, to treat acne vulgaris, comedo-type acne, polymorphic acne, acne rosacea, nodulocystic acne, acne conglobata, senile acne, secondary acne, solar acne, acne medicamentosa or occupational acne,
of ichthyosis, ichthyosiform states, Darrier's disease, keratosis palmaris and plantaris, leukoplasia and leukoplasiform states, cutaneous or mucosal (buccal) lichen,
of dermatological conditions with an inflammatory immunoallergic component, with or without cell proliferation disorder, of cutaneous, mucosal or ungual psoriasis, psoriatic rheumatism, or cutaneous atopy, eczema or respiratory atopy or gingival hypertrophy,
of dermal or epidermal proliferations whether benign or malignant, whether of viral origin or not, of verruca vulgaris, verruca plana and epidermodysplasia verruciformis, oral or florid papillomatoses, T lymphoma,
of proliferations induced by ultraviolet radiation, of baso- and spinocellular epithelioma,
of precancerous skin lesions, keratoacanthomas,
of immune dermatoses, lupus erythematosus,
of bullous immune diseases,
of collagen diseases, scleroderma,
of dermatological or general conditions having an immunological component,
of skin disorders due to exposure to UV radiation, of skin aging, photoinduced or chronological or actinic pigmentations and keratoses, or any pathologies associated with chronological or actinic aging, xerosis,
of sebaceous function disorders, acne hyperseborrhoea, simple seborrhoea or seborrhoeic dermatitis,
of cicatrization disorders or of stretch marks,
of pigmentation disorders, hyperpigmentation, melasma, hypopigmentation or vitiligo,
of lipid metabolism conditions, obesity, hyperlipidaemia or non-insulin-dependent diabetes or X syndrome,
of inflammatory conditions, arthritis,
of cancerous or precancerous states,
of alopecia of different origins, of alopecia due to chemotherapy or to radiation,
of immune system disorders, of asthma, diabetes mellitus type 1, multiple sclerosis, or other selective dysfunctions of the immune system, or
of conditions of the cardiovascular system, arteriosclerosis or hypertension,
comprising administering to an individual in need of such treatment, a thus effective amount of the cosmetic/pharmaceutical composition as defined by claim 33.

40. The cosmetic/pharmaceutical composition as defined by claim 33, formulated as a tablet, capsule, syrup, suspension, powder, granule, or emulsion.

41. The cosmetic/pharmaceutical composition as defined by claim 33, formulated as a salve, cream, milk, ointment, pad, gel, spray, mousse, lotion, stick, shampoo or washing base.
